SEQUENCE LISTING (72 sequences)

SEQ ID NO: 1 - protein, 148 aa, agrobacterium
MSQSEKSTSSDIVDRREGPKIEGFKVVSTRLRSAEYESFSRQARLLGLSDSMAIRVAVRRIAGFLEIDAETRQMMEAILHSIGALSNNIAALLCAYAENPTTDLGALQAERNAFGKSFADLDGLLRSILSVSRRRIDGCSMLADALQH

SEQ ID NO: 2 - protein, 147 aa, Agrobacterium
MSQGSRPTSSDIAVNQRECVKVEGFKVVSTRLRSAEYESFSHQARLLGLSDSMAIRVAVRRIGGFLEIDAETRHRMEAILQSIGTLSSNIAALLSAYAENPTMDLEALRAERIAFGKSFADLDGLLRSILSVSRRRIDGCSLLKDAL

SEQ ID NO: 3 - protein, 147 aa, Agrobacterium
MSKHTRVTSSETAINQHRSLNVEGFKVVSARLRSAEYETFSYQARLLGLSDSMAIRVAVRRIGGFLEIDAHTREKMEAILQSIGILSSNVSMLLSAYAEDPRSDLEAVRDERIAFGEAFAALDGLLRSILSVSRRRIDGCSLLKGAL

SEQ ID NO: 4 - protein, 147 aa, Agrobacterium; identical to SEQ ID NO: 3

SEQ ID NO: 5 - protein, 147 aa, Agrobacterium
MSQGSKPTSSDIAINQRVGATVEGFRVVSTRLRSAEYESFSHQARLLGLSDSMAIRVAVRRIGGFLEIDAETRHKMEAILLSIGTLSSNIAALLSAYAENPTMDLEALRAERIAFGESFADLDGLLRSILSVSRRRIDGCSMLKDSL

SEQ ID NO: 6 - protein, 109 aa, Agrobacterium
MSQGSRPTSSDIAVNQRECVKVEGFKVVSTRLRSAEYESFSHQARLLGLSDSMAIRVPVRRIGGFLEIDAETRHRMEAILQSIGTLSSNIAALLSAYAENPTMDLSFAS

SEQ ID NO: 7 - protein, 147 aa, Agrobacterium
MSQSAQPTSSDIDRRESPKVEGFKVVSTRLRSAEYESFSRQARLLGLSDSMAIRVAVRRIGGFLEIDAETRQKMEAILYSIGALSSNVAALLCAYAENPIPDLDALQAERNAFGKSFADLDALLRSILSVSRRRIDGCAMLREASQY

SEQ ID NO: 8 - protein, 147 aa, Agrobacterium
MSKHTRATSSETTINQHRSLKVEGFKVVSARLRSAEYETFSYQARLLGLSDSMAIRVAVRRIGGFLEIDADTREKMEAILQSIGILSSNVSMLLSAYAEDPRSDLEAVRDERIAFGEAFAALDGLLRSILSVSRRRIDGRSLLKGAL

SEQ ID NO: 9 - protein, 147 aa, Agrobacterium; identical to SEQ ID NO: 8

SEQ ID NO: 10 - protein, 148 aa, Agrobacterium
MSQSEKSTSSDIVDRREGPKIEGFKVVSTRLRSSEYESFSRQARLLGLSDSMAIRVAVRRIAGFLEIDAETRQMMEAILHSIGALSNNIAALLCAYAENPTTDLGALQAERNAFGKSFADLDGLLRSILSVSRRRIDGCSMLADALQH

SEQ ID NO: 11 - protein, 148 aa, Agrobacterium
MSQSAKSPSDNIVDRREGSTIEGFKVVSTRLRSAEYESFSRQARRLGLSDSMAIRIAVRRIAGFLEIDAETRHEMEAILHSIGTLSNNIAALLNSYAENPTTDLGALQAERNAFGKAFADLDGLLRSILSVSRRRIDGCSMLADASKH

SEQ ID NO: 12 - protein, 157 aa, Agrobacterium
MRVIEMTEDYSPNLFSDAPVRRRRSAAIVDPLGYKIVSVRLRLAEFESFSEQLRTLGLTNSLALRIAVRRIAGFLEIDAHTRQNLQEITSSIGQIADALNDTSRIASRDGRVDIERFESYRQQFGQEFAALDALLRTILNVSRRRQDGRRLLQESEQ

SEQ ID NO: 13 - protein, 448 aa, Agrobacterium
MPDRSQVIIRIVPGGGTKTLQQIINQFEYLSRKGKLELQRSARHLDIFVPPDEIRELARSWLQETGTYHESQPDEERQQDLTTHIIVSFPAGTSQVAAYAASREWAAEMFGSGAGGGRYNYLTAFHIDRDHPHLHVVVNRRELLGHGWLKISRRHPQLNYDALRIKMSEISLRHGVVLEATRRAERGITERPMTFAQYRRLERQQANQIRFEDPEFEEFSPGGNDREPDQSFNSSHGEPPQNVSVSSERNECLQPVSEARSRAPIGSNGNGVTPDRVPSENDSRSQSGSGEAFMDDDNLRNGPLTIAGDGQDLEGRSGIHRLATEPVTHTTSEDDVRQRPHRKRPRGDEEEQSGAKLTRVNGVRTGVTISDVPVAQDDPITSPIQPPGPNPLHDPGQTSIATDALPPTADRPQQGEPNSKRARDDDAEPSIRKRSRDGRSQGDEGNRR

SEQ ID NO: 14 - protein, 424 aa, Agrobacterium
MPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQAQKIQFEDTDFDETSPEEDRRDLSQSFDPFRSDPSTGEPDRATRHDKQPLEQHARFQESAGSSIKADARIRVSLESERSAQPSASKIPVIGHFGIETSYVAEASVRKRSGIFGTSRPVTDVAMHTVKRQQRSKRRNDEEAGPSGANRKGLKAAQVDSEANVGEQDTRDDSNKAADPVSASIGTEQPEASPKRPRDRHDGELGGRKRARGNRRDDGRGGT

SEQ ID NO: 15 - protein, 447 aa, Agrobacterium
MPDRAQVIIRIVPGGGTKTLQQIINQLEYLSRKGRLELQRSARHLDIPLPPDQIHELARSWVQETGTYDESQPDEERQQELTTHIIVSFPAGTSQVAAYAASREWAAEMFGSGAGGGRYNYLTAFHIDRDHPHLHVVVNRRELLGHGWLKISRRHPQLNYDALRIKMAEISLRHGIALDASRRAERGITERPITYAQYRRLEREQARQIRFEDADLEQSSPQGDHPEFSQPFDTSPFEASAGGPEDMPRPNNRQNESQVHLQEPAGVSNEAGVLVRVALETERLAQPFVSETILADDIGSGSSRVAEGRVESANRTPDIPRAATEAATHTTHDRQRRAKRPHDDDGGPSGAKRVTLEGIAVGPQANAGEQDGSSGPLVRQAGTSRPSPPTATTRASTATDSLSATAHLQQRRGVPSKRPREDDDGEPSERKRERDERSKDGREGNRR

SEQ ID NO: 16 - protein, 424 aa, Agrobacterium; identical to SEQ ID NO: 14

SEQ ID NO: 17 - protein, 436 aa, Agrobacterium
MPDRAQVIIRIVPGGGTKTLQQIINQLEYLSRKGKLELQRSARHLDIPLPPDQIHELARSWVQETGTYDESQPDEERQQELTTHIIVSFPAGTSQAAAYAASREWAAEMFGSGAGGGSYNYLTAFHIDRDHPHLHVVVNRRELLGHGWLKISRRHPQLNYDALRINMAEISLRHGIVLDASSRAERGIFERPITYAQFRRLERQARQIRFEDADLEQSSSQGDHPEFSQSPDTAPFEASAGRSEGMPYPNNRQNGSQVHLHEPAGFSNRAGGSVRIALETQRLAIFADDIESGSPPVSDVRAGNANADSDLPRSTVARTTDYSQRWSKRPRDDDEGPSGAKRVRLEGMAVGPEANAGERDSRGDPVAPPAETSRPSSLQDMARPNTATDPLAASGHLEQRRGTLSKRPRVEDDGEPSERKRARDDRSQDGRGGNRR

SEQ ID NO: 18 - protein, 447 aa, Agrobacterium
MPDRAQVIIRIVPGGGTKTLQQIINQLEYLSRKGRLELQRSARHLDIPLPPDQIHELARSWVQETGTYDESQPDEERQQELTTHIIVSFPAGTSQVAAYAASREWAAEMFGSGAGGGRYNYLTAFHIDRDHPHLHVVVNRRELLGHGWLKISRRHPQLNYDALRIKMAEISLRHGIALDASRRAERGITERPITYAQYRRLEREQARQIRFEDADLEQSSPQGDHPEFSQPFDTSPFEASAGGPEDMPRPNNRQNESQVHLQEPAGVSNEAGVLVRVALETERLAQPFVSETILADDIGSGSSRVAEGRVESANRTPDIPRAATEAATHTTHDRQRRAKRPHDDDGGPSGAKRVTLEGIAVGPQRTAGEQDGSSGPLERQAGTSRPSPPTATTRASTATDSLSATAHLQQRRGVLSKRPREDDDGEPSERKRERDERSKDGRGGNRR

SEQ ID NO: 19 - protein, 447 aa, Agrobacterium
MPDRAQVIIRIMPGGGTKTLQQIINQLEYLSRKGRLELQRSARHLDIPLPPDQIHELARSWVQETGTYDESQPDEERQQELTTHIIVSFPAGTSQVAAYAASREWAAEMFGSGAGGGRYNYLTAFHIDRDHPHLHVVVNRRELLGHGWLKISRRHPQLNYDALRIKMAEISLRHGIALDASRRAERGITERPITYAQYRRLEREQARQIRFEDADLEQSSPQGDHPEFSQPFDTSPFEASAGGPEDMPRPNNRQNESQVHLQEPAGVSNEAGVLVRVALETERLAQPFVSETILADDIGSGSSRVAEGRVESANRTPDIPRAATEAATHTTHDRQRRAKRPHDDDGGPSGAKRVTLEGIAVGPQANAGEQDGSSGPLVRQAGTSRPSPPTATTRASTATDSLSATAHLQQRRGVLSKRPREDDDGEPSERKRERDERSKDGRGGNRR

SEQ ID NO: 20 - protein, 447 aa, Agrobacterium
MPDRAQVIIRIVPGGGTKTLQQIINQLEYLSRKGRLELQRSARHLDIPLPPDQIHELARSWVQETGTYDESQPDEERQQELTTHIIVSFPAGTSQVAAYAASREWAAEMFGSGAGGGRYNYLTAFHIDRDHPHLHVVVNRRELLGHGWLKISRRHPQLNYDALRIKMAEISLRHGIALDASRRAERGITERPITYAQYRRLEREQARQIRFEDADLEQSSPQGDHPEFSQPFDTSPFEASAGGPEDMPRPNNRQNESQVHLQEPAGVSNEAGVLVRVALETERLAQPFVSETILADDIGSGSSRVAEGRVESANRTPDIPRAATEAATHTTHDRQRRAKRPHDDDGGPSGAKRVTLEGIAVGPQANAGEQDGSSGPLVRQAGTSRPSPPTATTRASTATDSLSATAHLQQRRGVLSKRPREDDDGEPSERKRERDERSKDGRGGNRR

SEQ ID NO: 21 - protein, 242 aa, Agrobacterium
MPERAQVIIRIVPGGGTKSLQQIINQLEYLSRKGKLELQRSARHLDVFVPPDEIRALAKSWVQETGTYDESRPDEERQQELTTHIIVSFPAGTSHAAAYAASREWAAEMHGSGAGGGLYNYLTAFHIDRDHPHLHVVVNRRELLGHGWLKISRRHPQLNYDAMRIKMADISLRHGIMLDATRRAERGITERPITYAQYRRLEREQANQIRFEDSDLEQLSSGGNQQELDRSSVLLSVSHPET

SEQ ID NO: 22 - protein, 424 aa, Agrobacterium
MPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGQGWLKISRRHPQLNYDGLRKKMAEISLRHGIVLDATSRAERGIAERPITYAEYRRLERMQAQKIQFEDTDFDETSPEEDRRDLSQSFDPFRSDASAGEPDRATRHDKQPLEPHARFQEPAGSSIKADARIRVPLESERGAQPSASKIPVTGHFGIETSYVAEASVPKQSGNSDTSRPVTDVAMHTVERQQRSKRRHDEEAGPSGANRKRLKAAQVDSEANVGEPDGRDDSNKAADPVSASIRTEQPEASPTCPRDRHDGELGERKRARGNRRDDGRGGT

SEQ ID NO: 23 - protein, 424 aa, Agrobacterium; identical to SEQ ID NO: 22

SEQ ID NO: 24 - protein, 448 aa, Agrobacterium
MPDRSQVIIRIVPGGGTKTLQQIINQLEYLSRKGKLELQRSARHLDTFVPPDEIRELALSWVQETGTYHESRPDEERQQELTTHIIVSFPAGTSQVAAYAASREWAAEMFGSGAGGGRYNYLTAFHIDRDHPHLHVVVNRRELLGHGWLKISRRHPQLNYDALRITMAEISLRHGVVLEATSRAERGISERPMTFAQYRRLERQQANQIRFEDIDFEEFSPGGNDGEPDQSFNSSYGSLPQNVSENLRRNGGLQSVSEVRSRALIGSNGNGVTPDRVSSGNDSRSQSGSDKAFVDDGNLRNGPLTIAGDGQDLEGRSGMHRLATEPVTHTTSEDDVRQRPHRKRPRGDEEEQSGAKLTRVDGIRTGVTISAVPVAQDDPITSPIQPPGSNPLDDPGQTNIATDALPPTADRRQQGEPNSKRPRDDDAEPSIRKRSRDGRSQGDEGNRR

SEQ ID NO: 25 - protein, 403 aa, Agrobacterium
MPDRSQVIIRIVPGGGTKTLQQIINQLEYLSRKGTLELQRSARHLDIFVPPDNIRDLARSWIQETGTYDESQPDEERQKELTTHIIVSFPAGTSQPAAYAASREWAAETFGSGEGGGQYNYLTAFHTDRDHPHLHVVVNRRELLGHKWLKISRRHPQLNYDALRTRMAKVSFRHGIVLEATRRAERGITERPMTFAQYRRLERQQANQIRFDDPDFEHFTPGENDREPEQFFDSSYGERPQNLPESPERNEHLQPMSEGRSRARIGSNGDGRDVEGASGTGLAIESVTHPTSEDDVQQRLHRKRPRGDEDEDRGAKLTRVNRVRAGVAITDVPVTGDGPTSSIQAPGSNPSQTDIATDGLPATADRPQQGGPNSKRPRDEEAEPSIRKRSRDGRSQEDEGNRR

SEQ ID NO: 26 - DNA, 35 bp, Agrobacterium tumefaciens
gctggtggcaggatatattgtggtgtaaacaaatt

SEQ ID NO: 27 - DNA, 35 bp, Agrobacterium tumefaciens
gtgtttgacaggatatattggcgggtaaacctaag

SEQ ID NO: 28 - DNA, 35 bp, Agrobacterium tumefaciens
agcggcggcaggatatattcaattgtaaatggctt

SEQ ID NO: 29 - DNA, 24 bp, Agrobacterium tumefaciens
ggcaggatatattcaattgtaaat

SEQ ID NO: 30 - DNA, 35 bp, Agrobacterium tumefaciens
ctgactggcaggatatataccgttgtaatttgagc

SEQ ID NO: 31 - DNA, 24 bp, Agrobacterium tumefaciens
ggcaggatatataccgttgtaatt

SEQ ID NO: 32 - DNA, 35 bp, Agrobacterium tumefaciens
actgatggcaggatatatgcggttgtaattcattt

SEQ ID NO: 33 - DNA, 35 bp, Agrobacterium tumefaciens
aaaggtggcaggatatatcgaggtgtaaaatatca

SEQ ID NO: 34 - DNA, 24 bp, Agrobacterium tumefaciens
cggcaggatatatggcagtgtaaa

SEQ ID NO: 35 - DNA, 2360 bp, Agrobacterium tumefaciens
gggatcagaagcaggtttgagctagcgatgattgtgttgttgagctcgctagagccgccacgcgtatcggccaacgcataatcaaatccttcgagctcggcattttcataggctgcctcaagaaggggcatttcgtcggcggaatagacttcacagcgaggatcccaggtactgctttgtaaggcgttttctctccatcgcgtcagaggccggttttcgtcggcatcgaagagggccactcgtttaccgtcatttgccaaagcagcgcaaaggcccatgagtgcggtggttttgccagcaccccctttgaaagagcaaaacgtcaaaagttgcatattctgatcccgcctgtcctgtgaaacggagtgcatttgtatttttgttcgtataaatgtttttgtgattatcgatgagtaaaagcgttgttacactattttttatttcaaattcgttataattaaattgcaattgtagcaattatattcggtttttcctgtaaatatactgttgatttcatatcgagtagggctagactttaatctgtctacccgggcacatttcgtgctggagtattcagaccttccgctttttttggaggaagctatgtcaaaacacaccagagtcacgtcgagtgagactgccatcaaccagcatcgatccctgaacgttgaagggtttaaggtcgtgagtgcccgtctgcgatcggccgagtatgaaaccttttcctatcaagcgcgcctgctgggactttcggatagtatggcaattcgcgttgcggtgcgtcgcatcgggggctttctcgaaatagatgcacacacccgagaaaagatggaagccatacttcagtccatcggaatactctcaagtaatgtatccatgcttctatctgcctacgccgaagaccctcgatcggatctggaggctgtgcgagatgaacgtattgcttttggtgaggctttcgccgccctcgatggcctactccgctccattttgtccgtatcccggcgacggatcgacggttgctcgctattgaaaggtgccttgtagcacttgaccacgcacctgacgggagaaaattggatgcccgatcgcgctcaagtaatcattcgcattgtgccaggaggtggaaccaagacccttcagcagataatcaatcagttggagtacctgtcccgtaagggaaagctggaactgcagcgttcagcccggcatctcgatattcccgttccgccggatcaaatccgtgagcttgcccaaagctgggttacggaggccgggatttatgacgaaagtcagtcagacgatgataggcaacaagacttaacaacacacattattgtaagcttccccgcaggtaccgaccaaaccgcagcttatgaagccagccgggaatgggcagccgagatgtttgggtcaggatacgggggtggccgctataactatctgacagcctaccacgtcgaccgcgatcatccacatttacatgtcgtggtcaatcgtcgggaacttctggggcacgggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaacatcgccgccttgagcggatgcaggctcaaaagattcaattcgaagatacagattttgatgagacctcgcctgaggaagatcgtcgggacctcagtcaatcgttcgatccatttcgatcggacccatctaccggcgaaccggaccgtgcaacccgacatgacaaacaaccgcttgaacagcacgcccgtttccaggagtccgccggctccagcatcaaagccgacgcacggatccgcgtatcattggagagcgagcggagtgcccaaccatccgcgtccaaaatccctgtaattgggcatttcgggattgagacttcctatgtcgctgaagccagcgtgcgcaaacgaagcggcattttcggtacttctcgcccggtgactgacgttgccatgcacacagtcaagcgccagcagcgatcaaaacgacgtaatgacgaggaggcaggtccgagcggagcaaaccgtaaaggattgaaggctgcgcaagttgattccgaggcaaatgtcggtgagcaagacactcgcgatgacagcaacaaggcggctgatccggtgtctgcttccatcggtaccgagcaaccggaagcttctccaaagcgtccgcgtgaccgtcacgatggagaattgggtggacgcaaacgtgcaagaggtaatcgtcgcgacgatgggcgcggggggacctagagaca

SEQ ID NO: 36 - DNA, 28 bp, Artificial sequence
  primer
aaactagtgggatcagaagcaggtttga

SEQ ID NO: 37 - DNA, 26 bp, artificial sequence
  primer
aaactagttgtctctaggtccccccg

SEQ ID NO: 38 - DNA, 15 bp, artificial sequence
  primer
ggccgcgcagccacc

SEQ ID NO: 39 - DNA, 26 bp, artificial sequence
  primer
caaatgcactccgtttcacaggacag

SEQ ID NO: 40 - DNA, 20 bp, artificial sequence
  primer
tgttcggttccctctaccaa

SEQ ID NO: 41 - DNA, 22 bp, artificial sequence
  primer
caacatccatcaccttgactga

SEQ ID NO: 42 - DNA, 24 bp, artificial sequence
  primer
cacagaaccgtcgcttcagcaaca

SEQ ID NO: 43 - DNA, 25 bp, artificial sequence
  primer
gccaattttagacaagtatcaaacg

SEQ ID NO: 44 - DNA, 24 bp, artificial sequence
  primer
ggcaggatatattcaattgtaaat

SEQ ID NO: 45 - DNA, 23 bp, artificial sequence
  primer
attgacgcttagacaacttaata

SEQ ID NO: 46 - DNA, 27 bp, artificial sequence
  primer
ctctttctttttctccatattgaccat

SEQ ID NO: 47 - DNA, 19 bp, artificial sequence
  primer
ggcttcatgtccgggaaat

SEQ ID NO: 48 - DNA, 19 bp, artificial sequence
  primer
atactcattgctgatccat

SEQ ID NO: 49 - DNA, 19 bp, artificial sequence
  primer
cgcgtgtcgaggaattctg

SEQ ID NO: 50 - DNA, 26 bp, artificial sequence
  primer
tctttatttcgacgtgtctacattca

SEQ ID NO: 51 - DNA, 14 bp, artificial sequence
  primer
ctggcccccatttg

SEQ ID NO: 52 - DNA, 21 bp, artificial sequence
  primer
gatggcgttgcgagagaagca

SEQ ID NO: 53 - DNA, 21 bp, artificial sequence
  primer
ggctcgattgtacctgcgttc

SEQ ID NO: 54 - DNA, 24 bp, artificial sequence
  primer
tccgtgagatcagccgacgcaccg

SEQ ID NO: 55 - DNA, 21 bp, artificial sequence
  primer
cgagcgggtgttccttcttca

SEQ ID NO: 56 - DNA, 22 bp, artificial sequence
  primer
catccgcttgccctcatctgtt

SEQ ID NO: 57 - DNA, 26 bp, artificial sequence
  primer
tgctcaacgggaatcctgctctgcga

SEQ ID NO: 58 - DNA, 17 bp, artificial sequence
  primer
cccgctcaagctggaaa

SEQ ID NO: 59 - DNA, 18 bp, artificial sequence
  primer
caatgcaccaggtcatca

SEQ ID NO: 60 - DNA, 15 bp, artificial sequence
  primer
cggcgaagcctgcga

SEQ ID NO: 61 - DNA, 19 bp, artificial sequence
  primer
gaccgtaaggcttgatgaa

SEQ ID NO: 62 - DNA, 21 bp, artificial sequence
  primer
cgagattctccgcgctgtaga

SEQ ID NO: 63 - DNA, 19 bp, artificial sequence
  primer
cttagctggataacgccac

SEQ ID NO: 64 - DNA, 25 bp, artificial sequence
  primer
caatcgaaatatgacgaaatggaaa

SEQ ID NO: 65 - DNA, 17 bp, artificial sequence
  primer
gcatgatccttcggctg

SEQ ID NO: 66 - DNA, 16 bp, artificial sequence
  primer
acgacatccgcctgct

SEQ ID NO: 67 - DNA, 19 bp, artificial sequence
  primer
gatgcctcagtgggaaagg

SEQ ID NO: 68 - DNA, 22 bp, artificial sequence
  primer
ccataggtgagagtggtgacaa

SEQ ID NO: 69 - DNA, 8 bp, artificial sequence
  primer
cttggagc

SEQ ID NO: 70 - DNA, 18 bp, artificial sequence
  primer
tggcggacgacgacttgt

SEQ ID NO: 71 - DNA, 19 bp, artificial sequence
  primer
aaagtttggaggctgccgt

SEQ ID NO: 72 - DNA, 21 bp, artificial sequence
  primer
cgagcagaccgccgtgtactt

(19) United States
(12) Patent Application Publication
(10) Pub. No.: US 2016/0319291 A1
(43) Pub. Date: Nov. 3, 2016

(54) USE OF AGROBACTERIUM VIR GENES IN PLASMIDS

(71) Applicant: Dow AgroSciences LLC, Indianapolis, IN (US)

(72) Inventors: Nagesh N. SARDESAI, West Lafayette, IN (US); Manju GUPTA, Carmel, IN (US); Huixia H. WU, Zionsville, IN (US); Stephen FOULK, Carmel, IN (US)

(21) Appl. No.: 14/963,501

(22) Filed: Dec. 9, 2015

Related U.S. Application Data
(60) Provisional application No. 62/091,239, filed on Dec. 12, 2014.

(51) Int. Cl. C12N 15/82; C12R 1/01; C12N 1/20

(52) U.S. Cl. CPC C12N 15/8205; C12N 1/20; C12R 1/01; C12N 15/8261; C12N 15/8274

(57) ABSTRACT

The present disclosure relates to a system of Agrobacterium-mediated plant transformation including plasmids containing transformation-enhancing genes within Agrobacterium strains. Further disclosed herein are Agrobacterium strains for use in plant transformation, and transgenic plants produced with the Agrobacterium strains.

### REFERENCE TO SEQUENCE LISTING SUBMITTED ELECTRONICALLY

The official copy of the sequence listing is submitted electronically via EFS-Web as an ASCII formatted sequence listing with a file named “73951USNP_ST25.txt”, created on Dec. 8, 2015, and having a size of 74,691 bytes, and is filed concurrently with the specification. The sequence listing contained in this ASCII formatted document is part of the specification, and is herein incorporated by reference in its entirety.

### FIELD OF THE INVENTION

The present disclosure relates to a system for Agrobacterium-mediated transformation of plants. The system disclosed herein includes plasmids that harbor transformation-enhancing genes within an Agrobacterium strain. Further disclosed herein are Agrobacterium strains for use in plant transformation, and transgenic plants produced with the Agrobacterium strains.

### BACKGROUND

Agrobacterium-mediated transformation of plants is a preferred method for introducing transgenic polynucleotide sequences into the plant genome. Over the last thirty years, this transformation technique has evolved and changed thereby resulting in numerous distinct systems, methodologies, and compositions for integrating a T-DNA region which contains a polynucleotide sequence into the genome of crop plants.

A. tumefaciens and A. rhizogenes are plant pathogenic soil bacteria known to be useful to genetically transform plant cells. The pTi and pRi plasmids of A. tumefaciens and A. rhizogenes, respectively, carry genes responsible for genetic transformation of the plant. Kado, C. I., Crit. Rev. Plant. Sci. 10:1 (1991). Descriptions of Agrobacterium vector systems and methods for Agrobacterium-mediated gene transfer are also available, for example, Gruber et al., supra, Miki et al., supra, Moloney et al., Plant Cell Reports 8:238 (1989), and U.S. Pat. Nos. 4,940,838 and 5,464,763.

If Agrobacterium is used for the transformation, the DNA to be inserted should be cloned into special plasmids, namely either into an intermediate vector or into a binary vector. Intermediate vectors cannot replicate themselves in Agrobacterium. The intermediate vector can be transferred into Agrobacterium tumefaciens by means of a helper plasmid (conjugation). The Japan Tobacco Superbinary system is an example of such a system (reviewed by Komari et al., (2006) In: Methods in Molecular Biology (K. Wang, ed.) No. 343: Agrobacterium Protocols (2^{nd} Edition, Vol. 1) HUMANA PRESS Inc., Totowa, N.J., pp. 15-41; and Komori et al., (2007) Plant Physiol. 145:1155-1160). Binary vectors can replicate, both in E. coli and in Agrobacterium and can be transformed directly into Agrobacterium. Typically these vectors comprise a selection marker gene and a linker or polylinker which are framed by the right and left T-DNA border regions. Additional T-DNA sequences for insertion into the plant genome may be included between the right and left T-DNA border regions. The Agrobacterium host cell comprises a plasmid (e.g., the pTi or pRi plasmid) comprising a vir region which is necessary for the transfer of the T-DNA region into a plant cell.

The virulence functions of the Agrobacterium tumefaciens host will direct the insertion of a T-strand (containing the construct and adjacent marker) into the plant cell DNA when the cell is infected by the bacteria using a binary T DNA vector (Bevan (1984) Nuc. Acid Res. 12:8711-8721) or the co-cultivation procedure (Horsch et al. (1985) Science 227:1229-1231). Generally, the Agrobacterium transformation system is used to engineer dicotyledonous plants (Bevan et al. (1982) Ann. Rev. Genet 16:357-384; Rogers et al. (1986) Methods Enzymol. 118:627-641). The Agrobacterium transformation system may also be used to transform, as well as transfer, DNA to monocotyledonous plants and plant cells. See U.S. Pat. No. 5,591,616; Hernalsteen et al. (1984) EMBO J 3:3039-3041; Hooykass-Van Slogteren et al. (1984) Nature 311:763-764; Grimsley et al. (1987) Nature 325:1677-179; Boulton et al. (1989) Plant Mol. Biol. 12:31-40; and Gould et al. (1991) Plant Physiol. 95:426-434. Following the introduction of the genetic construct into plant cells, plant cells can be grown and upon emergence of differentiating tissue such as shoots and roots, mature plants can be generated. In some embodiments, a plurality of plants can be generated. Methodologies for regenerating plants are known to those of ordinary skill in the art and can be found, for example, in: Plant Cell and Tissue Culture, 1994, Vasil and Thorpe Eds. Kluwer Academic Publishers and in: Plant Cell Culture Protocols (Methods in Molecular Biology 111, 1999 Hall Eds Humana Press). The genetically modified plant described herein can be cultured in a fermentation medium or grown in a suitable medium such as soil. In some embodiments, a suitable growth medium for higher plants can include any growth medium for plants, including, but not limited to, soil, sand, or any other particulate media that supports root growth (e.g., vermiculite, perlite, etc.) or hydroponic culture, as well as suitable light, water and nutritional supplements which optimize the growth of the higher plant.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, MacMillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al. (1987) Ann. Rev. of Plant Phys. 38:467-486.

Yet, despite the large amount of research activity in this area, there still remains a need to improve the efficiency of the Agrobacterium-mediated transformation process. Agrobacterium is a highly-complex system that functions with transformation-enhancing genes and specific DNA features that are located on autonomously replicating plasmids, and within the bacterial genome. Producing, a transgenic event that is full length, single copy, and free of any superfluous plasmid backbone sequences is desirable. Accordingly, it is desirable to integrate a T-strand into the genome of plants without any unwanted binary plasmid backbone being integrated within the plant genome.

Therefore, a need exists for systems, compositions and methods that can be utilized to improve the Agrobacterium-mediated transformation process to integrate a single, full length copy of the T-strand into the plant genome that is free of any residual binary vector backbone sequence. The development of a novel and improved Agrobacterium-mediated transformation system provides significant benefits for cell biologists.

The intensive screening efforts required for identifying plants that do not contain a plasmid backbone utilize significant labor and reagents. By eliminating the integration of the binary vector backbone within the plant genome, the costs associated with the production of transgenic plants can be reduced. Accordingly, the deployment of such a system which can result increased efficiency of the Agrobacterium-mediated transformation protocol is desirable. The present disclosure provides a novel and alternative approach for Agrobacterium-mediated plant transformation.

### BRIEF SUMMARY

The present invention is directed to a system for Agrobacterium-mediated transformation of a plant, wherein the system comprises three plasmids. In one embodiment, the first plasmid comprises a first virD1 coding sequence and a first virD2 coding sequence, the second plasmid comprises a nucleic acid sequence flanked at one end with a right Agrobacterium T-DNA border and at the other end with a left Agrobacterium T-DNA border, and the third plasmid comprises a second virD1 coding sequence and a second virD2 coding sequence, wherein the first virD1 and first virD2 originate from an Agrobacterium strain that is different from the Agrobacterium strain from which the second virD1 coding sequence and the second virD2 coding sequence originate. In one embodiment, the two different Agrobacterium strains differ in the opine they synthesize. In one embodiment, the first virD1 coding sequence and first virD2 coding sequence originate from an Agrobacterium strain that synthesizes octopine, and the second virD1 coding sequence and the second virD2 coding sequence originate from an Agrobacterium strain that synthesizes succinamopine. In one embodiment, the first virD1 coding sequence and first virD2 coding sequence originate from an Agrobacterium strain that synthesizes either nopaline or succinamopine and the second virD1 coding sequence and the second virD2 coding sequence originate from an Agrobacterium strain that synthesizes octopine. In a further embodiment, the second plasmid comprises a left and right border sequence that originates from the same Agrobacterium strain as the first virD1 coding sequence and first virD2 coding sequence of the first plasmid.

In one embodiment, the present disclosure provides, a system for Agrobacterium-mediated transformation of a plant comprising: a first plasmid comprising a first virD1 coding sequence and a first virD2 coding sequence, wherein the first virD1 coding sequence and the first virD2 coding sequence originate from a bacterial strain that synthesizes octopine; a second plasmid comprising an Agrobacterium T-DNA border, wherein the Agrobacterium T-DNA border originates from a bacterial strain that synthesizes octopine; and, a third plasmid comprising a second virD1 coding sequence and a second virD2 coding sequence, wherein the second virD1 coding sequence and the second virD2 coding sequence originate from a bacterial strain that synthesizes succinamopine.

Furthermore, the present disclosure provides an Agrobacterium strain selected from the group consisting of a nopaline synthesizing strain, a mannopine synthesizing strain, a succinamopine synthesizing strain, or an octopine synthesizing strain. In an embodiment, the strain comprises a first plasmid comprising a first virD1 coding sequence and a first virD2 coding sequence, wherein the first virD1 coding sequence and the first virD2 coding sequence originate from a bacterial strain that synthesizes octopine, a second plasmid comprising an Agrobacterium T-DNA border, wherein the Agrobacterium T-DNA border originates from a bacterial strain that synthesizes octopine, and a third plasmid comprising a second virD1 coding sequence and a second virD2 coding sequence, wherein the second virD1 coding sequence and the second virD2 coding sequence originate from a bacterial strain that synthesizes succinamopine.

In addition, the present disclosure provides a transgenic plant or plant cell produced by contacting plant cells with an Agrobacterium strain of the present disclosure. In an embodiment, said strain comprises a first plasmid comprising a first virD1 coding sequence and a first virD2 coding sequence, wherein the first virD1 coding sequence and the first virD2 coding sequence originate from a bacterial strain that synthesizes octopine, a second plasmid comprising an Agrobacterium T-DNA border, wherein the Agrobacterium T-DNA border originates from a bacterial strain that synthesizes octopine, and a third plasmid comprising a second virD1 coding sequence and a second virD2 coding sequence, wherein the second virD1 coding sequence and the second virD2 coding sequence originate from a bacterial strain that synthesizes succinamopine. In another embodiment, the disclosure provides for selecting plant cells that have a T-DNA region integrated into plant genome and regenerating plants from said cells. Further embodiments include transgenic plants with reduced binary vector backbone integration. As such, the reduction of binary vector backbone integration is determined by comparing transgenic plants of the subject disclosure to transgenic plants produced using other transformation methods (e.g., transgenic plants produced from alternative transformation methods such as other Agrobacterium-mediated transformation methods). In additional embodiments, the transgenic plant encodes an agronomic trait. In other embodiments, the transgenic plant produces a commodity product.

### BRIEF DESCRIPTION OF THE FIGURES

FIG. 1 provides an alignment between VirD1 protein sequences obtained from Agrobacterium that have been disclosed in the NCBI genbank. In addition, the pTi15955 (octopine) and pTiBo542 (succinamopine) VirD1 protein sequences are provided.

FIG. 2 provides an alignment between VirD2 protein sequences obtained from Agrobacterium that have been disclosed in the NCBI genbank. In addition, the pTi15955 (octopine) and pTiBo542 (succinamopine) VirD2 protein sequences are provided.

FIG. 3 provides an alignment of T-DNA border sequences obtained from octopine, nopaline, and succinamopine strains of Agrobacterium.

FIG. 4 provides an alignment between VirD2 protein sequences from pTi15955 (octopine) and pTiBo542 (succinamopine). There is 94% sequence identity between the two sequences.

FIG. 5 provides an alignment between VirD1 protein sequences from pTi15955 (octopine) and pTiBo542 (succinamopine). There is 96.6% sequence identity between the two sequences.

FIG. 6 provides an alignment between the T-DNA border sequences obtained from pTi15955 (octopine) and pTiBo542 (succinamopine).

FIG. 7 provides a Vector NTI™ map of pDAB112807. A polynucleotide fragment comprised of the virD promoter, virD1 and virD2 sequences were incorporated into existing plasmid pDAB9292 to produce pDAB112807.

FIG. 8 provides a schematic showing the relative positions of the binary vector backbone that were used for the design of molecular confirmation assays to detect the integration of the vector backbone sequences within the plant genome.

FIG. 9 provides a JMP graph showing the transformation frequency of Z. mays c.v. B104 infected with a ternary Agrobacterium strain (Reg Ter) and the Agrobacterium strain modified to include additional virD sequences (VirD).

FIG. 10 provides a JMP graph showing the frequency of transgenic events with vector backbone (BB) in Z. mays c.v. B104 infected with a ternary Agrobacterium strain (Reg Ter) and the Agrobacterium strain modified to include additional virD sequences (VirD).

FIG. 11 provides a JMP graph showing the frequency of 1-copy events in Z. mays c.v. B104 infected with a ternary Agrobacterium strain (Reg Ter) and the Agrobacterium strain modified to include additional virD sequences (VirD).

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “plant” includes a whole plant and any descendant, cell, tissue, or plant part. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

A plant cell is the structural and physiological unit of the plant. Plant cells, as used herein, includes protoplasts and protoplasts with a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant part” in embodiments herein.

The term “dicot” or “dicotyledonous” refers to plants having two cotyledons. Examples include crop plants such as soybean, sunflower, cotton, canola, rape, and mustard.

The term “monocot” or “monocotyledonous” refers to plants having a single cotyledon. Examples include crop plants such as maize, rice, wheat, oat, and barley.

The term “transgenic” refers to a cell or organism comprising a transgene, for example a “transgenic plant” refers to a plant comprising a transgene, i.e., a nucleic acid molecule artificially incorporated into the organism's genome as a result of human intervention.

The term “transgenic event” in reference to a plant refers to a recombinant plant produced by transformation and regeneration of a single plant cell with heterologous DNA, for example, an expression cassette that includes a transgene of interest. The term event refers to the original transformant and/or progeny of the transformant that includes the heterologous DNA. The term event also refers to progeny produced by a sexual outcross between the transformant and another plant. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected. In embodiments of the subject disclosure the particular event comprises a gene expression cassette polynucleotide inserted within a genomic locus.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

As used herein, “endogenous sequence” defines the native form of a polynucleotide, gene or polypeptide in its natural location in the organism or in the genome of an organism.

The term “isolated” as used herein means having been removed from its natural environment.

The term “purified,” as used herein relates to the isolation of a molecule or compound in a form that is substantially free of contaminants normally associated with the molecule or compound in a native or natural environment. The term further relates to a molecule or compound that has been increased in purity as a result of being separated from other components of the original composition. The term “purified nucleic acid” is used herein to describe a nucleic acid sequence which has been separated from other compounds including, but not limited to polypeptides, lipids and carbohydrates.

The terms “polypeptide,” “peptide,” and “protein” are used interchangeably to refer to a polymer of amino acid residues. The term also applies to amino acid polymers in which one or more amino acids are chemical analogues or modified derivatives of a corresponding naturally-occurring amino acids.

The term “exogenous sequence” or “heterologous sequence” as used herein is any nucleic acid sequence that has been removed from its native location and inserted into a new location altering the sequences that flank the nucleic acid sequence that has been moved. For example, an exogenous DNA sequence may comprise a sequence from another species.

A “fusion” molecule is a molecule in which two or more subunit molecules are linked, for example, covalently. The subunit molecules can be the same chemical type of molecule, or can be different chemical types of molecules.

For the purposes of the present disclosure, a “gene,” includes a DNA region encoding a gene product (see infra), as well as all DNA regions which regulate the production of the gene product, whether or not such regulatory sequences are adjacent to coding and/or transcribed sequences. Accordingly, a gene may include, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, interfering RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

Sequence identity: The term “sequence identity” or “identity,” as used herein in the context of two nucleic acid or polypeptide sequences, refers to the residues in the two sequences that are the same when aligned for maximum correspondence over a specified comparison window.

As used herein, the term “percentage of sequence identity” refers to the value determined by comparing two optimally aligned sequences (e.g., nucleic acid sequences, and amino acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10. The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (BLASTn) program may be employed using the default parameters. Nucleic acid sequences with even greater similarity to the reference sequences will show increasing percentage identity when assessed by this method.

As used herein, the terms “specifically hybridizable” and “specifically complementary” are terms that indicate a sufficient degree of complementarity, such that stable and specific binding occurs between the nucleic acid molecule and a target nucleic acid molecule under non-stringent conditions.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ and/or Mg++ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 3rd ed., vol. 1-3, Cold Spring Harbor Laboratory Press, Cold Spring Harbor, N.Y., 2001, chapters 9, 10 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology-Hybridization with Nucleic Acid Probes, Part I, Chapter 2, Elsevier, N Y, 1993; and Ausubel et al., Eds., Current Protocols in Molecular Biology, Chapter 2, Greene Publishing and Wiley-Interscience, N Y, 1995.

As used herein, “moderate stringency” conditions are those under which molecules with more than 20% sequence mismatch will not hybridize; conditions of “high stringency” are those under which sequences with more than 10% mismatch will not hybridize; and conditions of “very high stringency” are those under which sequences with more than 5% mismatch will not hybridize. The following are representative, non-limiting hybridization conditions.

High Stringency condition (detects sequences that share at least 90% sequence identity): Hybridization in 5×SSC and 0.1% SDS buffer at 65° C. for 16 hours; wash twice in 2×SSC and 0.1% SDS buffer at room temperature for 15 minutes each; and wash twice in 0.5×SSC and 0.1% SDS buffer at 65° C. for 20 minutes each.

Moderate Stringency condition (detects sequences that share at least 80% sequence identity): Hybridization in 5×-6×SSC and 0.1% SDS buffer at 65-70° C. for 16-20 hours; wash twice in 2×SSC and 0.1% SDS buffer at room temperature for 5-20 minutes each; and wash twice in 1×SSC and 0.1% SDS buffer at 55-70° C. for 30 minutes each.

Non-stringent control condition (sequences that share at least 50% sequence identity will hybridize): Hybridization in 6×SSC and 0.1% SDS buffer at room temperature to 55° C. for 16-20 hours; wash at least twice in 2×-3×SSC and 0.1% SDS buffer at room temperature to 55° C. for 20-30 minutes each.

As used herein, the term “substantially homologous” or “substantial homology,” with regard to a contiguous nucleic acid sequence, refers to contiguous nucleotide sequences that hybridize under stringent conditions to the reference nucleic acid sequence. For example, nucleic acid sequences that are substantially homologous to a reference nucleic acid sequence are those nucleic acid sequences that hybridize under moderate stringent conditions to the reference nucleic acid sequence. Substantially homologous sequences have at least 80% sequence identity. For example, substantially homologous sequences may have from about 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; about 99.9%, and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired.

As used herein “homologous” is used to refer to the relationship of a first gene to a second gene by descent from a common ancestral DNA sequence. The term, homolog, indicates a relationship between genes separated by the event of speciation (see ortholog) or to the relationship between genes separated by the event of genetic duplication (see paralog).

As used herein, the term “ortholog” (or “orthologous”) refers to a gene in two or more species that has evolved from a common ancestral nucleotide sequence, and may retain the same function in the two or more species.

As used herein, the term “paralogous” refers to genes related by duplication within a genome. Orthologs retain the same function in the course of evolution, whereas paralogs evolve new functions, even if these new functions are unrelated to the original gene function.

As used herein, two nucleic acid sequence molecules are said to exhibit “complete complementarity” when every nucleotide of a sequence read in the 5′ to 3′ direction is complementary to every nucleotide of the other sequence when read in the 3′ to 5′ direction. A nucleotide sequence that is complementary to a reference nucleotide sequence will exhibit a sequence identical to the reverse complement sequence of the reference nucleotide sequence. These terms and descriptions are well defined in the art, and are easily understood by those of ordinary skill in the art.

When determining the percentage of sequence identity between amino acid sequences, it is well-known by those of skill in the art that the identity of the amino acid in a given position provided by an alignment may differ without affecting desired properties of the polypeptides comprising the aligned sequences. In these instances, the percent sequence identity may be adjusted to account for similarity between conservatively substituted amino acids. These adjustments are well-known and commonly used by those of skill in the art. See, e.g., Myers and Miller (1988) Computer Applications in Biosciences 4:11-7. Statistical methods are known in the art and can be used in analysis of the identified 5,286 optimal genomic loci.

As used herein, the term “operably linked” refers to a linkage between two moieties that establishes a functional relationship between the two moieties. For example two amino acid sequences can be operably linked, or two nucleotide sequence can be operably linked, to form a contiguous sequence wherein the first sequence imparts functionality to the second. For instance, a promoter is operably linked to a coding sequence if the promoter affects the transcription or expression of the coding sequence. When recombinantly produced, operably linked nucleotide sequences are generally contiguous and, where necessary to join two protein-coding regions, in the same reading frame. However, nucleotide sequences need not be contiguous to be operably linked.

The term, “operably linked,” when used in reference to a regulatory sequence and a coding sequence, means that the regulatory sequence affects the expression of the linked coding sequence. “Regulatory sequences,” “regulatory elements,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule. In a further example, a right and left T-DNA border when operably linked to a T-DNA sequence will allow the transfer of the T-DNA from a plasmid to another location.

When used in reference to two or more amino acid sequences, the term “operably linked” means that the first amino acid sequence is in a functional relationship with at least one of the additional amino acid sequences.

As used herein, the term “transformation” or “transforming” refers to the transfer and integration of a nucleic acid or fragment thereof into a host organism, resulting in genetically stable inheritance. Host organisms containing a transforming nucleic acid are referred to as “transgenic,” “recombinant,” or “transformed” organisms. Known methods of transformation include, for example: Agrobacterium-mediated transformation (e.g., using a Agrobacterium tumefaciens, Agrobacterium rhizogenes, or another Agrobacterium bacterial strain to transform the plant material); calcium phosphate transformation; polybrene transformation; electroporation; ultrasonic methods (e.g., sonoporation); liposome transformation; microinjection; transformation with naked DNA; transformation with plasmid vectors; transformation with viral vectors; biolistic transformation (e.g., microparticle bombardment); silicon carbide WHISKERS™-mediated transformation; aerosol beaming; and PEG-mediated transformation.

The terms “plasmid” and “vector,” as used herein are interchangeable and refer to an extra chromosomal element that may carry one or more gene(s) that are not part of the central metabolism of the cell. Plasmids and vectors typically are circular double-stranded DNA molecules. However, plasmids and vectors may be linear or circular nucleic acids, of a single- or double-stranded DNA or RNA, and may be derived from any source, in which a number of nucleotide sequences have been joined or recombined into a unique construction that is capable of introducing a promoter fragment and a coding polynucleotide sequence along with any appropriate 3′ untranslated sequence into a cell. In examples, plasmids and vectors may comprise autonomously replicating sequences, genome integrating sequences, and/or phage or nucleotide sequences.

The term “Agrobacterium strain” or “strain” relates to components of the Agrobacterium organism other than plasmids or vectors (e.g., binary vector, pTi plasmid, or ternary plasmid). Thus, the bacterium is identified by the same strain name regardless of the presence or absence of any vector or plasmid.

The term “T-DNA border” refers to the left and right border sequences. The Left and Right border sequences are the target of endonuclease activity from the VirD gene product. The Right border is processed first to produce a T-strand. In addition, ‘overdrive’ sequences promote T-DNA transfer of the nopaline type Ti plasmid.

The term “gene expression cassette” refers to a nucleic acid construct comprising a heterologous nucleic acid which encodes a polypeptide under the control of a promoter, and terminated by a 3′-UTR.

The term “selectable marker” refers to a gene or polynucleotide whose expression allows identification of cells that have been transformed with a DNA construct or vector containing the gene or polynucleotide. Non-limiting examples of selectable markers include herbicide tolerance, antibiotic resistance, and visual reporter markers.

The term “synthesis” or “synthesize,” refers to formation of a particular chemical compound from its constituent parts using enzymatic synthesis processes. In some embodiments, the enzymes necessary for synthesis of a chemical compound are encoded within the Agrobacterium strain (i.e., opine synthesizing coding sequences). The term “catabolism” or “catabolize,” refers to the breakdown of complex substances into more simple ones with release of energy. In some embodiments, the enzymes necessary for catabolism of a chemical compound are encoded within the Agrobacterium strain (i.e., opine catabolizing coding sequences). When referring to specific opine synthesis of Agrobacterium, it is understood that such strains will also possess enzymes necessary for catabolism of the specific opine (e.g., an Agrobacterium strain that synthesizes nopaline is understood to also catabolize nopaline; an Agrobacterium strain that synthesizes succinamopine is understood to also catabolize succinamopine; an Agrobacterium strain that synthesizes octopine is understood to also catabolize octopine; and etc.).

As used herein in reference to a nucleic acid sequence, the phrase “originates from Agrobacterium strain” or “originates from bacterial strain” encompasses all known sequences that have been isolated from that strain. For example, a virD1 coding sequence originating from an Agrobacterium bacterial strain that synthesizes octopine encompasses all known virD1 coding sequences that have been isolated from such an Agrobacterium strain. Likewise, a virD2 coding sequence originating from an Agrobacterium bacterial strain that synthesizes octopine encompasses all known virD2 coding sequences that have been isolated from such an Agrobacterium strain. In addition, an Agrobacterium T-DNA border sequence originating from an Agrobacterium bacterial strain that synthesizes octopine encompasses all known Agrobacterium T-DNA border sequences that have been isolated from such an Agrobacterium strain.

The term “derivative,” as used herein, refers to a modification of a sequence set forth in the present disclosure. Illustrative of such modifications would be the substitution, insertion, and/or deletion of one or more bases relating to a nucleic acid sequence of a coding sequence or an operon within a plasmid/vector disclosed herein that preserve, slightly alter, or increase the function of a coding sequence disclosed herein in bacterial species. Such derivatives can be readily determined by one skilled in the art, for example, using computer modeling techniques for predicting and optimizing sequence structure. The term “derivative” thus also includes nucleic acid sequences having substantial sequence identity with the disclosed coding sequences herein such that they are able to have the disclosed functionalities for use in producing embodiments of the present disclosure.

As used herein, the phrase “binary vector system” refers to cloning vectors that are capable of replicating in both E. coli and Agrobacterium tumefaciens. In a binary vector system, two different plasmids are employed for generating transgenic plants. In some embodiments, the first plasmid is a smaller vector known as a “binary vector.” The binary vector refers to a plasmid that has an origin of replication (ori) that permits the maintenance of the plasmid in a wide range of bacteria including E. coli and Agrobacterium, contains foreign DNA within the T-DNA, contains T-DNA border sequences (or at least the right hand T-border sequence), markers for selection and maintenance in both E. coli and A. tumefaciens, and in some embodiments the binary vector may include a selectable marker for selection in plants. In many embodiments, the second plasmid is known as helper pTi plasmid, harbored in A. tumefaciens, which lacks the entire T-DNA region (i.e., a disarmed pTi plasmid) but contains an intact vir region essential for transfer of the T-DNA from Agrobacterium to plant cells.

### EMBODIMENTS

In one embodiment, the subject disclosure relates to a system for Agrobacterium-mediated transformation of a plant. More particularly, disclosed herein are Agrobacterium strains carrying DNA constructs that facilitate the transformation of plant cells as well as methods of using such strains to produce transgenic plants.

In accordance with one embodiment a system for conducting Agrobacterium mediated transformation of plants is provided comprising a first, second and third plasmid. In one embodiment, an Agrobacterium strain is provided comprising the first, second and third plasmid. The first, second and third plasmids are present in the Agrobacterium strain as self-replicating plasmids that comprise additional gene sequences for the transfer of a T-DNA from Agrobacterium into the genome of a plant cell. In one embodiment, the first plasmid comprises a first virD1 coding sequence and a first virD2 coding sequence, wherein the first virD1 coding sequence and the first virD2 coding sequence originate from a bacterial strain that synthesizes octopine. In one embodiment, the second plasmid comprises an Agrobacterium T-DNA border, wherein the Agrobacterium T-DNA border originates from a bacterial strain that synthesizes octopine. In one embodiment, the third plasmid comprises a second virD1 coding sequence and a second virD2 coding sequence, wherein the second virD1 coding sequence and the second virD2 coding sequence originate from a bacterial strain that synthesizes succinamopine. In further embodiments, plant cells with a T-DNA integrated into the plant genome are produced using an Agrobacterium strain comprising a first, second and third plasmid, as disclosed herein. In subsequent embodiments, the plant cells are selected to regenerate plants from said cells. In further embodiments of the disclosure, the T-DNA contains a gene expression cassette that encodes an agronomic trait. In additional embodiments, the agronomic trait produces a commodity product.

Agrobacterium species contain vir coding sequences that are involved in bacterial exopolysaccharide synthesis, maturation, and secretion. The vir coding sequences are located within the Agrobacterium genome, and on the pTi/pRi plasmid. In other aspects, the vir coding sequences are a collection of genes whose collective function is to excise the T-DNA region and promote its transfer, and integration into the plant genome.

The vir genes are necessary for transfer of the T-DNA region. The expression of the vir genes are induced by signals produced by plants following wounding. Phenolic compounds, such as acetosyringone, activate the virA gene, which is a constitutively expressed trans-membrane protein. The activated virA gene acts as a kinase, phosphorylating the virG gene. In its phosphorylated form, virG acts as an activator of transcription for the remaining vir gene operons. Activation of vir gene expression results in the generation of site-specific nicks within the bottom strand of all T-DNA border repeats, and a linear single-stranded DNA molecule (the T-strand) corresponding to the bottom strand of the T-DNA region.

The virD1 (helicase) and virD2 (endonuclease) coding sequences express proteins that form a dimer, which makes single-stranded cuts within the left and right borders that flank the T-DNA (i.e., T-DNA borders). The VirD2 protein remains covalently attached to the 5′ end of the T-strand complex. Next, VirE2 acts as a single stranded DNA binding protein, and this protein encases the T-strand complex and protects the single strand T-DNA region during the transport of the T-strand complex from the Agrobacterium strain into the plant cell. Once inside the plant cell, the VirD2 and VirE2 proteins dissociate from the T-DNA, and the complementary strand of the T-DNA is produced and integrated within the genome of the plant.

### The First DNA Plasmid

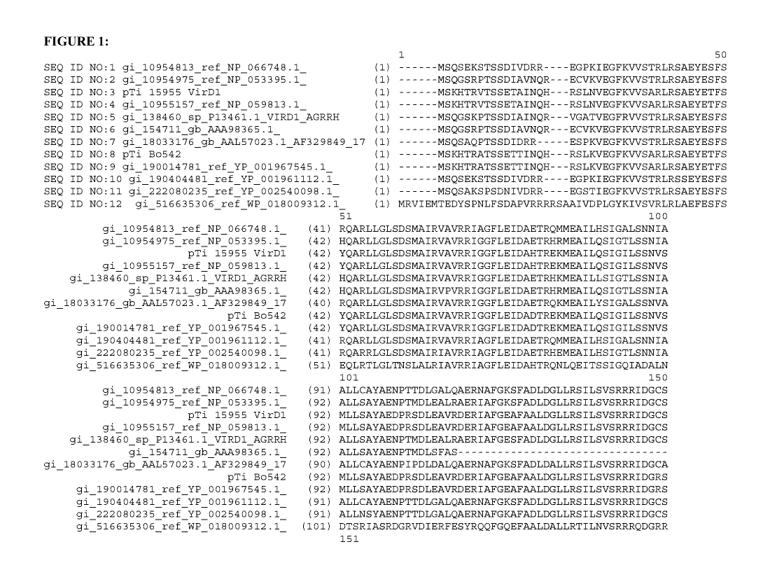

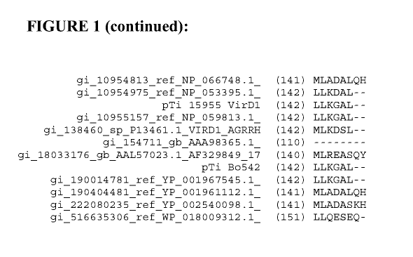

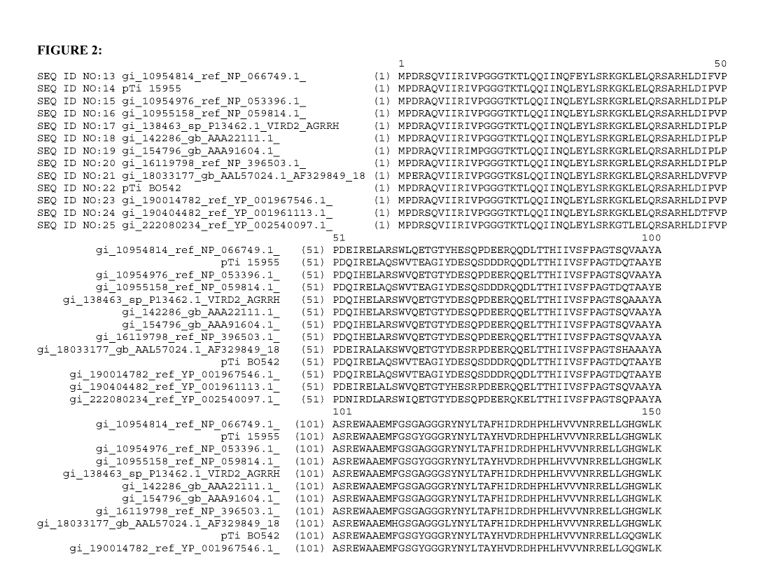

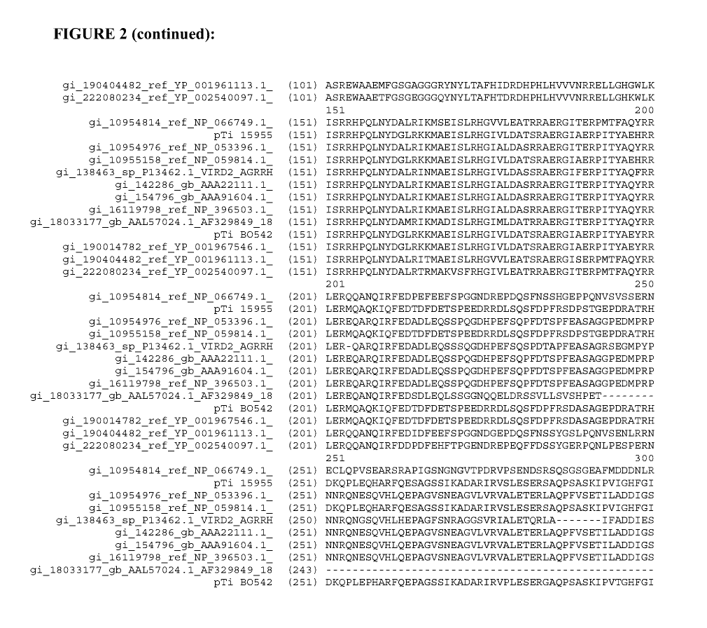

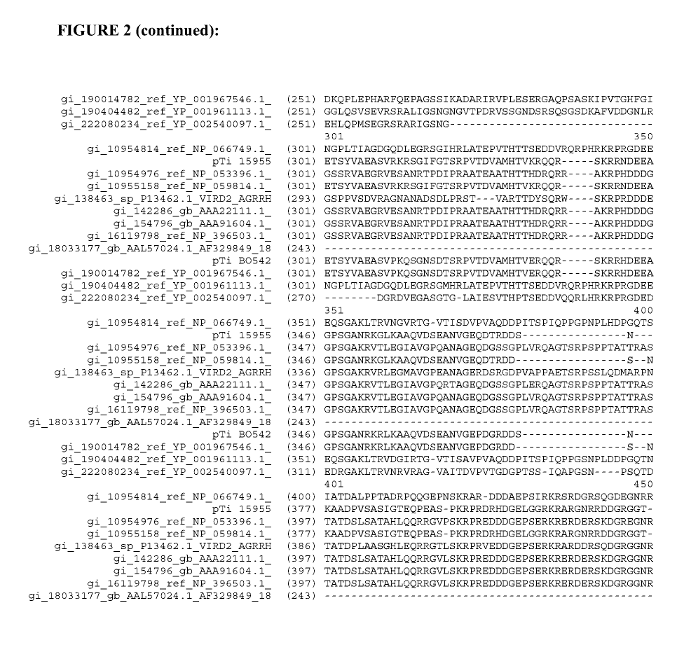

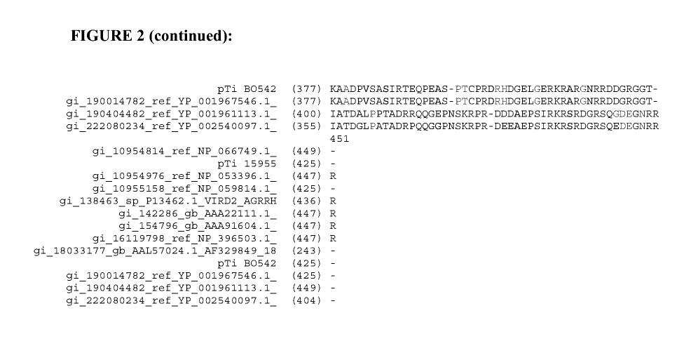

In an aspect of the embodiment, the system for Agrobacterium-mediated transformation of a plant comprises a first and a second virD1 coding sequence, and a first and second virD2 coding sequence. Agrobacterium has speciated into numerous strains that differ from one another in part based on the vir gene sequences which encode enzymes that are divergent from one another. In one embodiment, a system for Agrobacterium-mediated transformation of a plant is provided wherein the system comprises a first and a second virD1 coding sequence, and a first and second virD2 coding sequence, wherein the virD1 and virD2 coding sequences are independently selected from any of the virD1 and virD2 sequences known to encode proteins that are functional in transport of the T-strand complex from the Agrobacterium strain into the plant cell. Provided herein as FIG. 1 and FIG. 2 are VirD protein sequences presented as an alignment of VirD1 and VirD2 proteins, respectively. Further embodied in the subject disclosure are protein sequences that share 50%, 55%, 60%, 65%, 70%, 75%, 80%, 85%, 90%, 92.5%, 95%, 97.5%, 99%, 99.5%, or 99.9% sequence identity with the VirD1 sequences disclosed in FIG. 1. In additional embodiments of the subject disclosure are protein sequences that share 50%, 55%, 60%, 65%, 70%, 75%, 80%, 85%, 90%, 92.5%, 95%, 97.5%, 99%, 99.5%, or 99.9% sequence identity with the VirD2 sequences disclosed in FIG. 2.

In accordance with one embodiment any nucleic acid sequence encoding for a VirD1 and VirD2 protein as disclosed herein can be used in the Agrobacterium-mediated transformation system disclosed herein. For example the VirD1 encoding nucleic acid sequence may encode the VirD1 protein sequence of SEQ ID NO:1, or a sequence that shares at least 70%, 75%, 80%, 85%, 90%, 92.5%, 95%, 97.5%, 99%, 99.5%, or 99.9% sequence identity with SEQ ID NO:1. In an embodiment the VirD1 protein sequence comprises the sequence of SEQ ID NO:2, or a sequence that shares at least 70%, 75%, 80%, 85%, 90%, 92.5%, 95%, 97.5%, 99%, 99.5%, or 99.9% sequence identity with SEQ ID NO:2. In an embodiment, the VirD1 protein sequence comprises the sequence of SEQ ID NO:3 or a sequence that shares at least 70%, 75%, 80%, 85%, 90%, 92.5%, 95%, 97.5%, 99%, 99.5%, or 99.9% sequence identity with SEQ ID NO:3. In an embodiment, the VirD1 protein sequence comprises the sequence of SEQ ID NO:4, or a sequence that shares at least 70%, 75%, 80%, 85%, 90%, 92.5%, 95%, 97.5%, 99%, 99.5%, or 99.9% sequence identity with SEQ ID NO:4. In an embodiment, the VirD1 protein sequence comprises the sequence of SEQ ID NO:5, or a sequence that shares at least 70%, 75%, 80%, 85%, 90%, 92.5%, 95%, 97.5%, 99%, 99.5%, or 99.9% sequence identity with SEQ ID NO:5. In an embodiment, the VirD1 protein sequence comprises the sequence of SEQ ID NO:6, or a sequence that shares at least 70%, 75%, 80%, 85%, 90%, 92.5%, 95%, 97.5%, 99%, 99.5%, or 99.9% sequence identity with SEQ ID NO:6. In an embodiment, the VirD1 protein sequence comprises the sequence of SEQ ID NO:7, or a sequence that shares. at least 70%, 75%, 80%, 85%, 90%, 92.5%, 95%, 97.5%, 99%, 99.5%, or 99.9% sequence identity with SEQ ID NO:7. In an embodiment, the VirD1 protein sequence comprises the sequence of SEQ ID NO:8, or a sequence that shares at least 70%, 75%, 80%, 85%, 90%, 92.5%, 95%, 97.5%, 99%, 99.5%, or 99.9% sequence identity with SEQ ID NO:8. In an embodiment, the VirD1 protein sequence comprises the sequence of SEQ ID NO:9, or a sequence that shares at least 70%, 75%, 80%, 85%, 90%, 92.5%, 95%, 97.5%, 99%, 99.5%, or 99.9% sequence identity with SEQ ID NO:9. In an embodiment, the VirD1 protein sequence comprises the sequence of SEQ ID NO:10, or a sequence that shares at least 70%, 75%, 80%, 85%, 90%, 92.5%, 95%, 97.5%, 99%, 99.5%, or 99.9% sequence identity with SEQ ID NO:10. In an embodiment, the VirD1 protein sequence comprises the sequence of SEQ ID NO:11, or a sequence that shares at least 70%, 75%, 80%, 85%, 90%, 92.5%, 95%, 97.5%, 99%, 99.5%, or 99.9% sequence identity with SEQ ID NO:11. In an embodiment, the VirD1 protein sequence comprises the sequence of SEQ ID NO:12, or a sequence that shares at least 70%, 75%, 80%, 85%, 90%, 92.5%, 95%, 97.5%, 99%, 99.5%, or 99.9% sequence identity with SEQ ID NO:12. In one embodiment the VirD1 encoding nucleic acid sequence of the transformation system encodes a protein selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and an amino acid having at least 95% sequence identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

Furthermore, the transformation system may include a VirD2 coding sequence wherein the sequence encodes the VirD2 sequence of SEQ ID NO:13 or a protein sequence that shares at least 70%, 75%, 80%, 85%, 90%, 92.5%, 95%, 97.5%, 99%, 99.5%, or 99.9% sequence identity with SEQ ID NO:13. In an embodiment, the VirD2 protein sequence comprises the sequence of SEQ ID NO:14, or a sequence that shares at least 70%, 75%, 80%, 85%, 90%, 92.5%, 95%, 97.5%, 99%, 99.5%, or 99.9% sequence identity with SEQ ID NO:14. In an embodiment, the VirD2 protein sequence comprises the sequence of SEQ ID NO:15, or a sequence that shares at least 70%, 75%, 80%, 85%, 90%, 92.5%, 95%, 97.5%, 99%, 99.5%, or 99.9% sequence identity with SEQ ID NO:15. In an embodiment, the VirD2 protein sequence comprises the sequence of SEQ ID NO:16, or a sequence that shares at least 70%, 75%, 80%, 85%, 90%, 92.5%, 95%, 97.5%, 99%, 99.5%, or 99.9% sequence identity with SEQ ID NO:16. In an embodiment, the VirD2 protein sequence comprises the sequence of SEQ ID NO:17, or a sequence that shares at least 70%, 75%, 80%, 85%, 90%, 92.5%, 95%, 97.5%, 99%, 99.5%, or 99.9% sequence identity with SEQ ID NO:17. In an embodiment, the VirD2 protein sequence comprises the sequence of SEQ ID NO:18, or a sequence that shares at least 70%, 75%, 80%, 85%, 90%, 92.5%, 95%, 97.5%, 99%, 99.5%, or 99.9% sequence identity with SEQ ID NO:18. In an embodiment, the VirD2 protein sequence comprises the sequence of SEQ ID NO:19, or a sequence that shares at least 70%, 75%, 80%, 85%, 90%, 92.5%, 95%, 97.5%, 99%, 99.5%, or 99.9% sequence identity with SEQ ID NO:19. In an embodiment, the VirD2 protein sequence comprises the sequence of SEQ ID NO:20, or a sequence that shares at least 70%, 75%, 80%, 85%, 90%, 92.5%, 95%, 97.5%, 99%, 99.5%, or 99.9% sequence identity with SEQ ID NO:20. In an embodiment, the VirD2 protein sequence comprises the sequence of SEQ ID NO:21, or a sequence that shares at least 70%, 75%, 80%, 85%, 90%, 92.5%, 95%, 97.5%, 99%, 99.5%, or 99.9% sequence identity with SEQ ID NO:21. In an embodiment, the VirD2 protein sequence comprises the sequence of SEQ ID NO:22, or a sequence that shares at least 70%, 75%, 80%, 85%, 90%, 92.5%, 95%, 97.5%, 99%, 99.5%, or 99.9% sequence identity with SEQ ID NO:22. In an embodiment, the VirD2 protein sequence comprises the sequence of SEQ ID NO:23, or a sequence that shares at least 70%, 75%, 80%, 85%, 90%, 92.5%, 95%, 97.5%, 99%, 99.5%, or 99.9% sequence identity with SEQ ID NO:23. In an embodiment, the VirD2 protein sequence comprises the sequence of SEQ ID NO:24, or a sequence that shares at least 70%, 75%, 80%, 85%, 90%, 92.5%, 95%, 97.5%, 99%, 99.5%, or 99.9% sequence identity with SEQ ID NO:24. In an embodiment, the VirD2 protein sequence comprises the sequence of SEQ ID NO:25, or a sequence that shares at least 70%, 75%, 80%, 85%, 90%, 92.5%, 95%, 97.5%, 99%, 99.5%, or 99.9% sequence identity with SEQ ID NO:25 is disclosed. In one embodiment the VirD2 encoding nucleic acid sequence of the transformation system encodes a protein selected from the group consisting of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and an amino acid having at least 95% sequence identity with SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

In other embodiments, the VirD1 protein sequences may be classified and referred to by the Agrobacterium strain from which they originate. In further embodiments, the VirD1 protein may originate from a nopaline synthesizing Agrobacterium strain. In other embodiments, the VirD1 protein may originate from an octopine synthesizing Agrobacterium strain. In additional embodiments, the VirD1 protein may originate from a succinamopine synthesizing Agrobacterium strain. As previously described, VirD1 protein sequences are divergent from one another. Those with ordinary skill in the art would recognize further VirD1 proteins suitable for use as disclosed herein that originate from a nopaline synthesizing Agrobacterium strain, an octopine synthesizing Agrobacterium strain, or a succinamopine synthesizing Agrobacterium strain.

As previously described above, Agrobacterium strains may be categorized by the type of opine that they synthesize (and/or catabolize). As such, specific polynucleotide sequences may be obtained from such an Agrobacterium strain. In some embodiments, a VirD1 enzyme is obtained from an Agrobacterium strain. In further embodiments, a gene encoding a VirD1 enzyme is obtained from an octopine synthesizing Agrobacterium strain. Provided herein as an embodiment of the disclosure are octopine VirD1 enzymes. Further embodied in the subject disclosure are protein sequences that share 97.5%, 99%, 99.5%, or 99.9% sequence identity with the octopine VirD1 enzyme. In an embodiment, a nucleic acid sequence is provided that encodes the VirD1 protein sequence of SEQ ID NO:3 or a protein sequence with at least 97.5%, 99%, 99.5%, or 99.9% sequence identity with SEQ ID NO:3.

In further embodiments, a gene encoding a VirD1 enzyme is obtained from an succinamopine synthesizing Agrobacterium strain. Provided herein as an embodiment of the disclosure are succinamopine VirD1 enzymes. Further embodied in the subject disclosure are nucleic acid sequences that encode for protein sequences that share 97.5%, 99%, 99.5%, or 99.9% sequence identity with the succinamopine VirD1 enzyme of SEQ ID NO:8.

In other embodiments, the VirD2 proteins may be identified and referred to by the Agrobacterium strain from which they originate. For example, the VirD2 protein may originate from a nopaline synthesizing Agrobacterium strain. In one embodiment, the VirD2 protein originates from an octopine synthesizing Agrobacterium strain. In additional embodiments, the VirD2 protein originates from a succinamopine synthesizing Agrobacterium strain. As previously described, the VirD2 protein sequences are divergent. However, by categorizing the VirD2 proteins by the Agrobacterium strains from which the protein encoding gene sequence originates, those with ordinary skill in the art would recognize further undisclosed VirD2 proteins suitable for use as disclosed herein that originate from a nopaline synthesizing Agrobacterium strain, an octopine synthesizing Agrobacterium strain, or a succinamopine synthesizing Agrobacterium strain.

In some embodiments, a VirD2 enzyme is obtained from an Agrobacterium strain. In further embodiments, a gene encoding a VirD2 enzyme is obtained from an octopine synthesizing Agrobacterium strain. Provided herein as an embodiment of the disclosure are octopine VirD2 enzymes. Further embodied in the subject disclosure are protein sequences that share 95.5%, 97.5%, 99%, 99.5%, or 99.9% sequence identity with the octopine VirD2 enzyme. In an embodiment, a nucleic acid sequence is provided that encodes the VirD2 protein sequence of SEQ ID NO:14 or a protein sequence with at least 97.5%, 99%, 99.5%, or 99.9% sequence identity with SEQ ID NO:14. In further embodiments, a gene encoding a VirD2 enzyme is obtained from a succinamopine synthesizing Agrobacterium strain. Provided herein as an embodiment of the disclosure are succinamopine VirD2 enzymes. Further embodied in the subject disclosure are protein sequences that share 95.5%, 97.5%, 99%, 99.5%, or 99.9% sequence identity with the succinamopine VirD2 enzyme. Further embodied in the subject disclosure are protein sequences that share 95.5%, 97.5%, 99%, 99.5%, or 99.9% sequence identity with the octopine VirD2 enzyme. In an embodiment, a nucleic acid sequence is provided that encodes the VirD2 protein sequence of SEQ ID NO:22 or a protein sequence with at least 97.5%, 99%, 99.5%, or 99.9% sequence identity with SEQ ID NO:22 is disclosed.

Wildtype Agrobacterium strains may be pathogenic, and in some instances infection of Agrobacterium in plant species may manifest as a crown gall tumor. Typically crown gall tumors produce opine compounds (e.g., octopine or nopaline). The different types of opine compounds that are produced within the crown gall tumors are incited by the Agrobacterium strain which infects the crown gall tumor. Moreover, the genes that encode enzymes necessary for opine biosynthesis are located as sequences encoded on the wildtype T-strand of Agrobacterium strains.

Accordingly, the different types of Agrobacterium strains are categorized by the specific set of opine that the strains synthesize (and/or catabolize). Although, nopaline and octopine are the most commonly known types of opines, and most strains of Agrobacterium are categorized as either opine or nopaline type strains, there are over 30 different known opines. In addition, most opines are categorized as nopaline- or octopine-like, and may be further considered as members of either the nopaline or octopine family. For example, succinamopine is an opine that in certain circumstances is categorized as a member of the nopaline family.

### The Second DNA Plasmid

In a further aspect of the embodiment, the system for Agrobacterium-mediated transformation of a plant comprises a second plasmid comprising an Agrobacterium T-DNA border. In some embodiments, the T-DNA region is operably-linked to the Agrobacterium T-DNA border, and in a further embodiment the T-DNA region further comprises a the first T-DNA border (e.g., a left border) operably linked at one end of the T-DNA region, and a second T-DNA border (e.g., a right border) operably linked to the opposite end of the T-DNA region. In some embodiments, the first and second T-DNA borders are independently selected from the group consisting of a nopaline Agrobacterium T-DNA border, an octopine Agrobacterium T-DNA border, a succinamopine Agrobacterium T-DNA border, or any combination thereof.

In further embodiments, the Agrobacterium T-DNA border is selected from the group consisting of a nopaline Agrobacterium T-DNA border, an octopine Agrobacterium T-DNA border, a succinamopine Agrobacterium T-DNA border, or any combination thereof. For example, the T-DNA border sequences may be repeated on a plasmid. In some embodiments, the left hand T-DNA border sequences are directly repeated on a plasmid. In further embodiments, the left hand T-DNA border sequences are repeated on a plasmid, wherein a spacer polynucleotide sequence is placed between the border sequences. In subsequent embodiments, the right hand T-DNA border sequences are directly repeated on a plasmid. In one embodiment the T-DNA border is defined by 25 bp imperfect direct repeats. In other embodiments, the right hand T-DNA border sequences are repeated on a plasmid, wherein a spacer polynucleotide sequence is placed between one or more of the repeated border sequences. In additional embodiments, the second plasmid further comprises a second T-DNA border, wherein said T-DNA border is operably linked to one end of the T-DNA region, and said second T-DNA border is operably linked to the other end of the T-DNA region.

In wildtype, virulent pTi plasmids there are “virulent T-DNA genes” and “oncogenic T-DNA genes” located on the T-DNA, which are flanked by the T-DNA borders. The virulent T-DNA genes are transferred from the Agrobacterium to the plant genome, thereby resulting in a pathogenic phenotype (e.g., opine synthesis genes). These virulent T-DNA genes alter the development and metabolism of the infected cells, but are not necessary for successful integration of the T-DNA. As such, they are dispensable when plant transformation vectors are designed, and the only features necessary for transfer of the T-DNA are the Left and Right T-DNA border sequences. Agrobacterium strains for use in plant transformation have been disarmed. As such, the virulent T-DNA genes and oncogenic T-DNA genes that alter the development and metabolism of the infected cells have been removed from the T-DNA region. In place of the virulent T-DNA genes and oncogenic T-DNA genes a “T-DNA region” is provided comprising other polynucleotide sequences for transfer and integration within plant genomic DNA as a T-DNA. In one embodiment the T-DNA region comprises a nucleotide polylinker comprising two or more restriction endonuclease recognitions sites to assist in the subsequent insertion of other nucleic acid sequences. In one embodiment, the T-DNA region includes one or more gene expression cassettes that express agronomic traits of commercial importance.

The T-DNA borders structurally define the functional T-DNA region.

Accordingly, these border sequences are recognized and cleaved by VirD1 and VirD2 proteins to excise the T-DNA region. Genetic analysis of the 25 bp border sequence has demonstrated that the sequences are polar in function. The right hand T-DNA border is required for initial cleavage of a T-DNA. As such the excision of a T-DNA originates from the initial cleavage of the DNA at the right hand T-DNA border, and proceeds with subsequent cleavage at the left hand T-DNA border. In some and cleave the T-DNA borders, and T-DNA read-through will occur. As such, a T-DNA that is processed will extend into the remainder of the plasmid (i.e., backbone of the binary vector). Accordingly instances, the VirD1 and VirD2 proteins fail to efficiently bind, the T-DNA may include undesirable sequences from the binary backbone, such as antibiotic selectable markers, bacterial origin of replication sequences, superfluous polynucleotide vector sequences, and other polynucleotide sequences that are necessary for bacterial replication and stability. It is desirable to develop systems, methods, and composition of matter that improves the efficiency of the cleavage by the VirD1 and VirD2 proteins at the T-DNA borders so that T-DNA read-through into the plasmid backbone does not occur.

FIG. 3 provides an alignment of known T-DNA border sequences. In one embodiment, a T-DNA border suitable for use in accordance with the present disclosure comprises a polynucleotide sequence of FIG. 3 or polynucleotide sequences that share 90%, 92.5%, 95%, 97.5%, 99%, 99.5%, or 99.9% sequence identity with a T-DNA border sequence of FIG. 3.

Further embodied in the subject disclosure are T-DNA border sequences of SEQ ID NO:26 or a T-DNA border sequence that shares 90%, 92.5%, 95%, 97.5%, 99%, 99.5%, or 99.9% sequence identity with SEQ ID NO:26. Further embodied in the subject disclosure are T-DNA border sequences of SEQ ID NO:27 or a T-DNA border sequence that shares 90%, 92.5%, 95%, 97.5%, 99%, 99.5%, or 99.9% sequence identity with SEQ ID NO:27. Further embodied in the subject disclosure are T-DNA border sequences of SEQ ID NO:28 or a T-DNA border sequence that shares 90%, 92.5%, 95%, 97.5%, 99%, 99.5%, or 99.9% sequence identity with SEQ ID NO:28. Further embodied in the subject disclosure are T-DNA border sequences of SEQ ID NO:29 or a T-DNA border sequence that shares 90%, 92.5%, 95%, 97.5%, 99%, 99.5%, or 99.9% sequence identity with SEQ ID NO:29. Further embodied in the subject disclosure are T-DNA border sequences of SEQ ID NO:30 or a T-DNA border sequence that shares 90%, 92.5%, 95%, 97.5%, 99%, 99.5%, or 99.9% sequence identity with SEQ ID NO:30. Further embodied in the subject disclosure are T-DNA border sequences of SEQ ID NO:31 or a T-DNA border sequence that shares 90%, 92.5%, 95%, 97.5%, 99%, 99.5%, or 99.9% sequence identity with SEQ ID NO:31. Further embodied in the subject disclosure are T-DNA border sequences of SEQ ID NO:32 or a T-DNA border sequence that shares 90%, 92.5%, 95%, 97.5%, 99%, 99.5%, or 99.9% sequence identity with SEQ ID NO:32. Further embodied in the subject disclosure are T-DNA border sequences of SEQ ID NO:33 or a T-DNA border sequence that shares 90%, 92.5%, 95%, 97.5%, 99%, 99.5%, or 99.9% sequence identity with SEQ ID NO:33. Further embodied in the subject disclosure are T-DNA border sequences of SEQ ID NO:34 or a T-DNA border sequence that shares 90%, 92.5%, 95%, 97.5%, 99%, 99.5%, or 99.9% sequence identity with SEQ ID NO:34.

In a further aspect of the embodiment, the system for Agrobacterium-mediated transformation of a plant comprises an Agrobacterium T-DNA border that originates from a bacterial strain that synthesizes octopine. In one embodiment the Agrobacterium first and second T-DNA border sequences are independently selected from SEQID NO: 28, SEQ IDNO: 29, SEQ ID NO: 30, SEQID NO: 31, SEQ ID NO: 32, SEQ ID NO: 33 and a sequence having at least 95% sequence identity with SEQID NO: 28, SEQ IDNO: 29, SEQ ID NO: 30, SEQID NO: 31, SEQ ID NO: 32 or SEQ ID NO: 33.

As previously described above, Agrobacterium strains may be categorized by the type of opine that they synthesize (and/or catabolize). As such, specific polynucleotide sequences may be obtained from such an Agrobacterium strain. In some embodiments, a T-DNA border polynucleotide sequence is obtained from an Agrobacterium strain. In further embodiments, a T-DNA border polynucleotide sequence is obtained from an octopine synthesizing Agrobacterium strain. Provided herein as an embodiment of the disclosure are octopine T-DNA border sequences. Further embodied in the subject disclosure are polynucleotide sequences that share 90%, 92.5%, 95%, 97.5%, 99%, or 99.5% sequence identity with the octopine T-DNA border sequences. Further embodied in the subject disclosure are octopine T-DNA border sequences of SEQ ID NO:29. In an embodiment of the subject disclosure are octopine T-DNA border sequences that share 90%, 92.5%, 95%, 97.5%, 99%, 99.5%, or 99.9% sequence identity with SEQ ID NO:29. Further embodied in the subject disclosure are octopine T-DNA border sequences of SEQ ID NO:31. In an embodiment of the subject disclosure are octopine T-DNA border sequences that share 90%, 92.5%, 95%, 97.5%, 99%, 99.5%, or 99.9% sequence identity with SEQ ID NO:31.

In other embodiments, the T-DNA border used in the second plasmid of the present disclosure includes any of the border sequences disclosed herein including for example: the octopine T-DNA border sequence of SEQ ID NO:28, the octopine T-DNA border sequence of SEQ ID NO:30, the octopine T-DNA border sequences of SEQ ID NO:32, the nopaline T-DNA border sequences of SEQ ID NO:33, the nopaline T-DNA border sequences of SEQ ID NO:26, or the nopaline T-DNA border sequences of SEQ ID NO:27.

Further embodied in the subject disclosure are succinamopine T-DNA border sequences of SEQ ID NO:34. In an embodiment of the subject disclosure are succinamopine T-DNA border sequences that share 90%, 92.5%, 95%, 97.5%, 99%, 99.5%, or 99.9% sequence identity with SEQ ID NO:34.

### The Third DNA Plasmid

In a further aspect of the embodiment, the system for Agrobacterium-mediated transformation of a plant comprises a third plasmid. In some embodiments, the third plasmid is a pTi plasmid. In other embodiments, the third plasmid is a pRi plasmid.

The pTi plasmid (also known as a helper plasmid) comprises the vir regions necessary for the production and transfer of the T-DNA region. Most pTi plasmids that are used in Agrobacterium strains for plant transformation are disarmed. Accordingly, the vir and one gene regions that are located within the T-strand of wildtype, virulent Agrobacterium strains have been removed or mutated. However, the T-DNA borders remain, and are modified to include a polynucleotide sequence between the right and left T-DNA borders. In an embodiment, a wildtype and virulent pTi plasmid that has been modified to rearrange, mutate, delete, add, invert, or translocate a polynucleotide sequence are referred herein as a pTi plasmid derivative. In an embodiment, the T-DNA region has been modified to contain at least one gene expression cassette expressing an agronomic trait. Such pTi-derived plasmids, having functional vir genes and lacking all or substantially all of the T-region and associated elements are provided herein as an embodiment.

In subsequent embodiments, the pTi plasmid is a pTiBo542 plasmid. In an embodiment, the pTi plasmid is a derivative of a pTiBo542 plasmid (Hood, E. E.; Helmer, G. C.; Fraley, R. T.; Chilton, M. D. The hypovirulence of Agrobacterium tumefaciens A281 is encoded in the region of PtiB0542 outside the T-DNA. J. Bacteriol. 168:1291-1301; 1986, herein incorporated by reference in its entirety. In subsequent embodiments, the pTi plasmid is a pTiC58 plasmid (Holsters et al., The Functional Organization of the Nopaline A. tumefaciens plasmid pTiC58. Plasmid 3(2); 212-230, 1980, herein incorporated by reference in its entirety). In an embodiment, the pTi plasmid is a derivative of a pTiC58 plasmid. In subsequent embodiments, the pTi plasmid is a pTiAch5 plasmid (Gielen, J.; De Beuckeleer, M.; Seurinck, J.; Deboeck F.; De Greve H.; Lemmers, M.; Van Montagu M.; Schell J. The Complete Nucleotide Sequence of the TL-DNA of the Agrobacterium tumefaciens plasmid pTiAch5. The EMBO Journal. 3(4):835-846; 1984, herein incorporated by reference in its entirety). In an embodiment, the pTi plasmid is a derivative of a pTiAch5 plasmid. In subsequent embodiments, the pTi plasmid is a pTiChry5 plasmid (Kovacs L. G.; Pueppke S. G. Mapping and Genetic Organization of pTiChry5, a Novel Ti Plasmid from a Highly Virulent Agrobacterium tumefaciens Strain, Mol Gen Genet 242(3):327-336, 1994, herein incorporated by reference in its entirety). In an embodiment, the pTi plasmid is a derivative of a pTiChry5 plasmid. In subsequent embodiments, the pTi plasmid is a pTi15995 plasmid (Barker, R. F., Idler, K. B., Thompson, D. V. and Kemp, J. D. Nucleotide sequence of the T-DNA region from the Agrobacterium tumefaciens octopine Ti plasmid pTi15955, Plant Mol. Biol. 2 (6), 335-350, 1983, herein incorporated by reference in its entirety). In an embodiment, the pTi plasmid is a derivative of a pTi15995 plasmid.

In a further embodiment of the subject disclosure a VirBCDG fragment (See, International Patent Publication No. 2012/016222, herein incorporated by reference its entirety) integrated within the first plasmid, second plasmid, third plasmid, or an Agrobacterium genomic DNA is provided.

The Japan Tobacco™ super binary system is a specialized example of the shuttle vector/homologous recombination system (reviewed by Komari et al., (2006) In: Methods in Molecular Biology (K. Wang, ed.) No. 343: Agrobacterium Protocols (2nd Edition, Vol. 1) HUMANA PRESS Inc., Totowa, N.J., pp. 15-41; and Komori et al., (2007) Plant Physiol. 145:1155-1160, the disclosure of which is expressly incorporated herein by reference). The Agrobacterium tumefaciens host strain employed with the superbinary system is LBA4404 (pSB1). Strain LBA4404 (pSB1) harbors two independently-replicating plasmids, pAL4404 and pSB1. pAL4404 is a Ti-plasmid-derived helper plasmid which contains an intact set of vir genes (from Ti plasmid pTiACH5), but which has no T-DNA region (and, thus no T-DNA left and right border repeat sequences). Plasmid pSB1 supplies an additional partial set of vir genes derived from pTiBo542; this partial vir gene set includes the virB operon, and the virC operon, as well as genes virG and virD1. One example of a shuttle vector used in the superbinary system is pSB11, which contains a cloning polylinker that serves as an introduction site for genes destined for plant cell transformation, flanked by right and left T-DNA border repeat regions. Shuttle vector pSB11 is not capable of independent replication in Agrobacterium, but is stably maintained as a co-integrant plasmid when integrated into pSB1 by means of homologous recombination between common sequences present on pSB1 and pSB11. Thus, the fully modified T-DNA region introduced into LBA4404 (pSB1) on a modified pSB11 vector is productively acted upon and transferred into plant cells by Vir proteins derived from two different Agrobacterium Ti plasmid sources (pTiACH5 and pTiBo542). The superbinary system has proven to be particularly useful in transformation of monocot plant species. See Hiei et at., (1994) Plant J. 6:271-282 and Ishida et at., (1996) Nat. Biotechnol. 14:745-750.

In a further aspect of the embodiment, the system for Agrobacterium-mediated transformation of a plant comprises a first or a second plasmid. In some embodiments, the first plasmid is a plasmid that autonomously replicates within an Agrobacterium strain. In other embodiments, the second plasmid is a plasmid that autonomously replicates within an Agrobacterium strain.

In some embodiments the first plasmid is a binary vector. In other embodiments the second plasmid is a binary vector. Non-limiting examples of binary vectors include; pBIN binary vector (Bevan M (1984) Binary Agrobacterium vectors for plant transformation. Nucleic Acids Res 12: 8711-872, herein incorporated by reference in its entirety), pGA binary vector (An G (1987) Binary Ti vectors for plant transformation and promoter analysis. Methods Enzymol 153: 292-305 An G, Watson B D, Stachel S, Gordon M P, Nester E W (1985) New cloning vehicles for transformation of higher plants. EMBO J 4: 277-284, herein incorporated by reference in its entirety), SEV binary vector (Fraley R T, Rogers S G, Horsch R B, Eichholtz D A, Flick J S, Fink C L, Hoffmann N L, Sanders P R (1985) The SEV system: a new disarmed Ti plasmid vector system for plant transformation. Biotechnology (N Y) 3: 629-635, herein incorporated by reference in its entirety), pEND4K binary vector (Klee H J, Yanofsky M F, Nester E W (1985) Vectors for transformation of higher plants. Biotechnology (N Y) 3: 637-642, herein incorporated by reference in its entirety), pBI binary vector (Jefferson R A, Kavanagh T A, BevanMW (1987) GUS fusions: b-glucuronidase as a sensitive and versatile gene fusion marker in higher plants. EMBO J 6:3901-3907, herein incorporated by reference in its entirety), pCIB 10 binary vector (Rothstein S J, Lahners K N, Lotstein R J, Carozzi N B, Jayne S M, Rice D A (1987) Promoter cassettes, antibiotic-resistance genes, and vectors for plant transformation. Gene 53: 153-161, herein incorporated by reference in its entirety), pMRK63 binary vector (Vilaine F, Casse-Delbart F (1987) A new vector derived from Agrobacterium rhizogenes plasmids: a micro-Ri plasmid and its use to construct a mini-Ri plasmid. Gene 55: 105-114, herein incorporated by reference in its entirety), pGPTV binary vector (Becker D (1990) Binary vectors which allow the exchange of plant selectable markers and reporter genes. Nucleic Acids Res 18: 203, herein incorporated by reference in its entirety), pCGN1547 binary vector (McBride K E, Summerfelt K R (1990) Improved binary vectors for Agrobacterium-mediated plant transformation. Plant Mol Biol 14: 269-276, herein incorporated by reference in its entirety), pART binary vector (Gleave A P (1992) A versatile binary vector system with a T-DNA organizational structure conducive to efficient integration of cloned DNA into the plant genome. Plant Mol Biol 20: 1203-1207, herein incorporated by reference in its entirety), pGKB5 binary vector (Bouchez D, Camilleri C, Caboche M (1993) A binary vector based on Basta resistance for in planta transformation of Arabidopsis thaliana. C R Acad Sci Ser III Sci Vie 316: 1188-1193, herein incorporated by reference in its entirety), pMJD80 binary vector (Day M J D, Ashurst J L, Dixon R A (1994) Plant expression cassettes forenhanced translational efficiency. Plant Mol Biol Rep 12: 347-357, herein incorporated by reference in its entirety), pMJD81 binary vector (Day M J D, Ashurst J L, Dixon R A (1994) Plant expression cassettes forenhanced translational efficiency. Plant Mol Biol Rep 12: 347-357, herein incorporated by reference in its entirety), pPZP binary vector (Hajdukiewicz P, Svab Z, Maliga P (1994) The small, versatile pPZP family of Agrobacterium binary vectors for plant transformation. Plant Mol Biol 25: 989-994, herein incorporated by reference in its entirety), pBINPLUS binary vector (van Engelen F A, Molthoff J W, Conner A J, Nap J P, Pereira A, Stiekema W J (1995) pBINPLUS: an improved plant transformation vector based on pBIN19. Transgenic Res 4: 288-290, herein incorporated by reference in its entirety), pRT100 binary vector (Uberlacker B, Werr W (1996) Vectors with rare-cutter restriction enzyme sites for expression of open reading frames in transgenic plants. Mol Breed 2: 293-295, herein incorporated by reference in its entirety), pCB binary vector (Xiang C, Han P, Lutziger I, Wang K, Oliver DJ (1999) A mini binary vector series for plant transformation. Plant Mol Biol 40: 711-717, herein incorporated by reference in its entirety), pGreen binary vector (Hellens R P, Edwards E A, Leyland N R, Bean S, Mullineaux P M (2000) pGreen: a versatile and flexible binary Ti vector for Agrobacteriummediated plant transformation. Plant Mol Biol 42: 819-832, herein incorporated by reference in its entirety), pPZP-RCS2 binary vector (Goderis I J W M, De Bolle M F C, Francois I E J A, Wouters P F J, Broekaert W F, Cammue B P A (2002) A set of modular plant transformation vectors allowing flexible insertion of up to six expression units. Plant Mol Biol 50: 17-27, herein incorporated by reference in its entirety), pMDC binary vector (Curtis M D, Grossniklaus U (2003) A gateway cloning vector set for highthroughput functional analysis of genes in planta. Plant Physiol 133: 462-469, herein incorporated by reference in its entirety), pRCS2 binary vector (Chung S M, Frankman E L, Tzfira T (2005) A versatile vector system for multiple gene expression in plants. Trends Plant Sci 10: 357-361, herein incorporated by reference in its entirety), pEarleyGate binary vector (Earley K W, Haag J R, Pontes O, Opper K, Juehne T, Song K, Pikaard C S (2006) Gateway-compatible vectors for plant functional genomics and proteomics. Plant J 45: 616-629, herein incorporated by reference in its entirety), pGWTAC binary vector (Chen Q J, Zhou H M, Chen J, Wang X C (2006) A Gateway-based platform for multigene plant transformation. Plant Mol Biol 62: 927-936, herein incorporated by reference in its entirety), pORE binary vector (Coutu C, Brandle J, Brown D, Brown K, Miki B, Simmonds J, Hegedus D D (2007) pORE: A modular binary vector series suited for both monocot and dicot plant transformation. Transgenic Res 16: 771-781, herein incorporated by reference in its entirety), pSITE binary vector (Chakrabarty R, Banerjee R, Chung S M, Farman M, Citovsky V, Hogenhout S A, Tzfira T, Goodin M (2007) pSITE vectors for stable integration or transient expression of autofluorescent protein fusions in plants: probing Nicotiana benthamiana-virus interactions. Mol Plant Microbe Interact 20: 740-750, herein incorporated by reference in its entirety), pMSP binary vector (Lee L Y, Kononov M E, Bassuner B, Frame B R, Wang K, Gelvin S B (2007) Novel plant transformation vectors containing the superpromoter. Plant Physiol 145: 1294-1300, herein incorporated by reference in its entirety), pCAMBIA binary vector (http://www.cambia.org/daisy/cambia/materials/vectors), and pGD binary vector (Goodin M M, Dietzgen R G, Schichnes D, Ruzin S, Jackson A O (2002) pGD vectors: versatile tools for the expression of green and red fluorescent protein fusions in agroinfiltrated plant leaves. Plant J 31: 375-383, herein incorporated by reference in its entirety). See, herein incorporated by reference in its entirety. Binary vectors generally contain a number of important features such as T-DNA border sequences, origins of replication that are functional in both Escherichia coli and Agrobacterium strains, antibiotic resistance genes that are compatible with other antibiotic resistance harbored by the pTi/pRi plamid and/or Agrobacterium genome, and other features that improve plant transformation efficiency (e.g., overdrive sequence). Further features of binary vectors are known to those having ordinary skill in the art, for example see, Lee and Gelvin (2008) Plant Physiology, 146; 325-332 (herein incorporated by reference) which discloses many of the above described features of binary plasmids/vectors.

In some embodiments the first plasmid is a ternary vector. In other embodiments the second plasmid is a ternary vector. A “ternary” (i.e., three-plasmid) vector wherein a copy of the constitutive mutant virGN54D gene from pTi15955 was co-resident on a pBBR1-derived plasmid in Agrobacterium tumefaciens strain LBA4404 that contained the disarmed pTi helper plasmid pAL4404 and a binary vector harboring genes for plant transformation has been described. See van der Fits et al., (2000) Plant Molec. Biol. 43:495-502, herein incorporated by reference in its entirety. Additional non-limiting examples of a ternary vector are described in further detail at European Patent Application No. 2042602A1 and U.S. Patent Application No. 2010/0132068A1 that describe cosmid binary vectors and “booster” plasmids that, when present in an Agrobacterium cell harboring a pTi helper plasmid, constitute further examples of ternary plasmid systems, herein incorporated by reference in its entirety. Finally, International Patent Application No. 2012016222A2 describes a ternary plasmid system for use in Agrobacterium, herein incorporated by reference in its entirety. In subsequent embodiments, the binary vector may contain a first or second VirD1 encoding sequence. In other embodiments, the binary vector may contain a first or second VirD2 encoding sequence.

In some embodiments, the first plasmid is a BIBAC vector. In other embodiments, the second plasmid is a BIBAC vector. BIBAC vectors designed to enable efficient transformation of large DNA fragments into plant and non-plant host cells can be used. See, e.g., U.S. Pat. No. 5,733,744, U.S. Pat. No. 5,977,439, and U.S. Patent Application No. 2002/0123100A1. Refinements to the BIBAC system have used the virG gene which was employed alone or in combination with the virE1 and virE2 genes from pTiA6 in the UIA143 RecA-deficient strain. See, e.g., Hamilton et al. (1996) Proc. Natl. Acad. Sci. 93:9975-9979; Hamilton (1997), Gene 200:107-116; Frary and Hamilton (supra). In subsequent embodiments, the BIBAC vector may contain a first or second VirD1 encoding sequence. In other embodiments, the BIBAC vector may contain a first or second VirD2 encoding sequence.

Plasmids are assigned to incompatibility groups (genotypic designation: inc; group designation: Inc) based on sequences contained in the plasmid. The inc determinant typically serves to prevent other plasmids of the same or related incompatibility group from coexisting in the same host, and helps maintain a certain copy number of the plasmid within the cell. See, e.g., Fernandez-Lopez, et al. (2006) FEMS Microbiol. Rev. 30:942-66; and Adamczyk and Jagura-Burdzy (2003) Acta Biochim. Pol. 50:425-53. Two plasmids are incompatible if either is less stable in the presence of the other than it was by itself. Competition for cell resources can result when two plasmids of the same incompatibility group are found in the same cell. Whichever plasmid is able to replicate faster, or provides some other advantage, will be represented to a disproportionate degree among the copies allowed by the incompatibility system. Surprisingly, plasmids can also be incompatible when they both possess the same functions for partitioning themselves into daughter cells.

Plasmids typically fall into only one of the many existing incompatibility groups. There are more than 30 known incompatibility groups. Plasmids belonging to incompatibility group IncP have been studied thoroughly and a large number of plasmids which derive from this IncP group have been constructed (Schmidhauser et al. (1988) Biotechnology 10:287-332). Exemplary plasmids containing the IncP incompatibility group include: pMP90RK, pRK2013, pRK290, pRK404, and pRK415. These plasmids may be maintained in numerous bacterial species including E. coli and Agrobacterium tumefaciens. Examples of other incompatibility groups include, but are not limited to; IncN, IncW, IncL/M, IncT, IncU, IncW, IncY, IncB/O, IncFII, IncII, IncK, IncCom9, IncFI, IncFII, IncFIII, IncHI1, IncHI2, IncX, IncA/C, IncD, IncFIV, IncFV/FO, IncFVI, IncH1 3, IncHI1, Inc12, IncI, IncJ, IncV, IncQ, and the like, including variants thereof, e.g., exhibiting substantial sequence or functional relationship.

In addition, a suitable plasmid used to transform plant cell using the methods described herein can contain a selectable marker gene encoding a protein that confers on the transformed plant cells resistance to an antibiotic or a herbicide. The individually employed selectable marker gene may accordingly permit the selection of transformed cells while the growth of cells that do not contain the inserted DNA can be suppressed by the selective compound. The particular selectable marker gene(s) used may depend on experimental design or preference, but any of the following selectable markers may be used, as well as any other gene not listed herein that could function as a selectable marker. Examples of selectable markers include, but are not limited to, genes that provide resistance or tolerance to antibiotics such as kanamycin, G418, hygromycin, bleomycin, and methotrexate, or to herbicides, such as phosphinothricin (bialaphos), glyphosate, imidazolinones, sulfonylureas, triazolopyrimidines, chlorosulfuron, bromoxynil, and DALAPON.

In an embodiment, the subject disclosure relates to an Agrobacterium strain. While certain examples of Agrobacterium strains are described herein, the functionality discussed could be moved to other Agrobacterium strains with the same criteria, e.g., other strains which contain the first, second, and third plasmid. Examples of other strains that could be used with the systems, composition, and methods described herein include, but are not limited to, Agrobacterium tumefaciens strain LBA4404, Agrobacterium tumefaciens strain AGL-0, Agrobacterium tumefaciens strain AGL-1, Agrobacterium tumefaciens strain GV3101::pMP90; Agrobacterium tumefaciens strain NT1 (pKPSF2); Agrobacterium tumefaciens strain C58, Agrobacterium tumefaciens strain C58-Z707, Agrobacterium tumefaciens strain Chry5, Agrobacterium rhizogenes strains, Agrobacterium tumefaciens strain EHA101, Agrobacterium tumefaciens strain EHA105, Agrobacterium tumefaciens strain ABI, Agrobacterium tumefaciens strain MOG101, and Agrobacterium tumefaciens strain T37.

In some embodiments, the Agrobacterium strain is selected from the group consisting of a nopaline synthesizing strain, a mannopine synthesizing strain, a succinamopine synthesizing strain, or an octopine synthesizing strain. In an embodiment, the nopaline synthesizing strains of Agrobacterium include strain C58 and strain ABI. In a further embodiment, the octopine synthesizing strain of Agrobacterium includes strain LBA4404. In an embodiment, the succinamopine synthesizing strains of Agrobacterium include AGL-1, and AGL-0. In an additional embodiment, the Agrobacterium strain of the subject disclosure includes a T-DNA region comprising a gene expression cassette. In further embodiments, the Agrobacterium strain includes the gene expression cassette that contains a selectable marker. In further embodiments, the Agrobacterium strain comprises a VirBCDG fragment integrated within the first plasmid, second plasmid, third plasmid, or an Agrobacterium genomic DNA. In subsequent embodiments, the Agrobacterium strain comprises the second plasmid which further comprises a second T-DNA border, wherein said T-DNA border is operably linked to one end of the T-DNA region, and said second T-DNA border is operably linked to the other end of the T-DNA region.

The Agrobacterium strains discussed herein can be used advantageously to introduce one or more genes into a plant, e.g., to provide individual or multiple agronomic traits into the genome of a plant (e.g., insecticidal resistance or herbicidal tolerance properties). For example, the Agrobacterium strains can be used to introduce one or more, two or more, three or more, four or more, five or more, or six or more transgenes into a plant. Using the Agrobacterium strains described herein, a polynucleotide containing a selectable marker gene sequences is inserted into a single location in the plant cell when the plant cell is transformed. In terms of the size of the T-DNA regions used to insert the genes, the T-DNA regions can be equal to or greater than 1,000 nucleotide base pairs, the T-DNA regions can be equal to or greater than 5,000 nucleotide base pairs, the T-DNA regions can be equal to or greater than 10,000 nucleotide base pairs, the T-DNA regions can be equal to or greater than 15,000 nucleotide base pairs, greater than or equal to 20,000 nucleotide base pairs, equal to or greater than 25,000 nucleotide base pairs, equal to or greater than 26,000 nucleotide base pairs, equal to or greater than 27,000 nucleotide base pairs, equal to or greater than 28,000 nucleotide base pairs, equal to or greater than 29,000 nucleotide base pairs, or equal to or greater than 30,000 nucleotide base pairs.

In further aspects of subject disclosure the systems, compositions, and methods disclosed herein relate to a transgenic plant or plant cell. In other embodiments, the transgenic plant or plant cell is produced by contacting plant cells with an Agrobacterium strain comprising a first, second and third plasmid. In one embodiment, the first, second and third plasmids are self-replicating plasmids. In one embodiment, the first plasmid comprises a first virD1 coding sequence and a first virD2 coding sequence, wherein the first virD1 coding sequence and the first virD2 coding sequence originate from a bacterial strain that synthesizes octopine. The second plasmid comprises an Agrobacterium T-DNA border, wherein the Agrobacterium T-DNA border originates from a bacterial strain that synthesizes octopine. The third plasmid comprises a second virD1 coding sequence and a second virD2 coding sequence, wherein the second virD1 coding sequence and the second virD2 coding sequence originate from a bacterial strain that synthesizes succinamopine. In further embodiments, plant cells with a T-DNA integrated into the plant genome are produced using an Agrobacterium strain comprising a first, second and third plasmid, as disclosed herein. In subsequent embodiments, the plant cells are selected to regenerate plants from said cells. In further embodiments of the disclosure, the T-DNA contains a gene expression cassette that encodes an agronomic trait. In additional embodiments, the agronomic trait produces a commodity product.

In an embodiment, the subject disclosure relates to the introduction of one or more gene expression cassettes which are inserted within the plant genome. In some embodiments the gene expression cassettes comprise a coding sequence. The coding sequence can encode, for example, a gene that confers an agronomic trait. In further embodiments, the agronomic trait is selected from the group consisting of an insecticidal resistance trait, herbicide tolerance trait, nitrogen use efficiency trait, water use efficiency trait, nutritional quality trait, DNA binding trait, and selectable marker trait. In additional embodiments, the agronomic traits are expressed within the plant. An embodiment of the subject disclosure includes a plant comprising one or more agronomic traits.

In some embodiments the transgenic plant comprises a gene expression cassette. Standard recombinant DNA and molecular cloning techniques for the construction of a gene expression cassette as used herein are well known in the art and are described, e.g., by Sambrook et al., Molecular Cloning: A Laboratory Manual, Second Edition, Cold Spring Harbor Laboratory Press, Cold Spring Harbor, N.Y. (1989); and by Silhavy et al., Experiments with Gene Fusions, Cold Spring Harbor Laboratory Press, Cold Spring Harbor, N.Y. (1984); and by Ausubel et al., Current Protocols in Molecular Biology, published by Greene Publishing Assoc. and Wiley-Interscience (1987).

A number of promoters that direct expression of a gene in a plant can be employed in a gene expression cassette. Such promoters can be selected from constitutive, chemically-regulated, inducible, tissue-specific, and seed-preferred promoters. The promoter used to direct expression of a nucleic acid depends on the particular application. For example, a strong constitutive promoter suited to the host cell is typically used for expression and purification of expressed proteins.

Non-limiting examples of preferred plant promoters include promoter sequences derived from A. thaliana ubiquitin-10 (ubi-10) (Callis, et al., 1990, J. Biol. Chem., 265:12486-12493); A. tumefaciens mannopine synthase (Δmas) (Petolino et al., U.S. Pat. No. 6,730,824); and/or Cassava Vein Mosaic Virus (CsVMV) (Verdaguer et al., 1996, Plant Molecular Biology 31:1129-1139). Other constitutive promoters include, for example, the core Cauliflower Mosaic Virus 35S promoter (Odell et al. (1985) Nature 313:810-812); Rice Actin promoter (McElroy et al. (1990) Plant Cell 2:163-171); Maize Ubiquitin promoter (U.S. Pat. No. 5,510,474; Christensen et al. (1989) Plant Mol. Biol. 12:619-632 and Christensen et al. (1992) Plant Mol. Biol. 18:675-689); pEMU promoter (Last et al. (1991) Theor. Appl. Genet. 81:581-588); ALS promoter (U.S. Pat. No. 5,659,026); Maize Histone promoter (Chabouté et al. Plant Molecular Biology, 8:179-191 (1987)); and the like.

Other useful plant promoters include tissue specific and inducible promoters. An inducible promoter is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory element may be exposed to an inducer by externally applying the inducer to the cell or plant such as by spraying, watering, heating or similar methods.

Any inducible promoter can be used in the embodiments of the instant disclosure. See Ward et al., Plant Mol. Biol. 22: 361-366 (1993). Exemplary inducible promoters include ecdysone receptor promoters (U.S. Pat. No. 6,504,082); promoters from the ACE1 system which respond to copper (Mett et al., Proc. Natl. Acad. Sci. 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)); Tet repressor from Tn10 (Gatz et al., Mol. Gen. Genet. 227: 229-237 (1991); or promoters from a steroid hormone gene, the transcriptional activity of which is induced by a glucocorticosteroid hormone, Schena et al., Proc. Natl. Acad. Sci. U.S.A. 88: 10421 (1991) and McNellis et al., (1998) Plant J. 14(2):247-257; the maize GST promoter, which is activated by hydrophobic electrophilic compounds that are used as pre-emergent herbicides (see U.S. Pat. No. 5,965,387 and International Patent Application, Publication No. WO 93/001294); and the tobacco PR-la promoter, which is activated by salicylic acid (see Ono S, Kusama M, Ogura R, Hiratsuka K., “Evaluation of the Use of the Tobacco PR-la Promoter to Monitor Defense Gene Expression by the Luciferase Bioluminescence Reporter System,” Biosci Biotechnol Biochem. 2011 Sep. 23; 75(9):1796-800). Other chemical-regulated promoters of interest include tetracycline-inducible and tetracycline-repressible promoters (see, for example, Gatz et al., (1991) Mol. Gen. Genet. 227:229-237, and U.S. Pat. Nos. 5,814,618 and 5,789,156).

Other regulatable promoters of interest include a cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84(19):6624-6628 (1987)), inducible by anaerobic conditions; and the light-inducible promoter derived from the pea rbcS gene or pea psaDb gene (Yamamoto et al., (1997) Plant J. 12(2):255-265); a light-inducible regulatory element (Feinbaum et al., Mol. Gen. Genet. 226:449, 1991; Lam and Chua, Science 248:471, 1990; Matsuoka et al. (1993) Proc. Natl. Acad. Sci. USA 90(20):9586-9590; Orozco et al. (1993) Plant Mol. Bio. 23(6):1129-1138), a plant hormone inducible regulatory element (Yamaguchi-Shinozaki et al., Plant Mol. Biol. 15:905, 1990; Kares et al., Plant Mol. Biol. 15:225, 1990), and the like. An inducible regulatory element also can be the promoter of the maize In2-1 or In2-2 gene, which responds to benzenesulfonamide herbicide safeners (Hershey et al., Mol. Gen. Gene. 227:229-237, 1991; Gatz et al., Mol. Gen. Genet. 243:32-38, 1994), and the Tet repressor of transposon Tn10 (Gatz et al., Mol. Gen. Genet. 227:229-237, 1991). Stress inducible promoters include salt/water stress-inducible promoters such as P5CS (Zang et al., (1997) Plant Sciences 129:81-89); cold-inducible promoters, such as, cor15a (Hajela et al., (1990) Plant Physiol. 93:1246-1252), cor15b (Wilhelm et al., (1993) Plant Mol Biol 23:1073-1077), wsc1 (Ouellet et al., (1998) FEBS Lett. 423-324-328), ci7 (Kirch et al., (1997) Plant Mol Biol. 33:897-909), ci21A (Schneider et al., (1997) Plant Physiol. 113:335-45); drought-inducible promoters, such as Trg-31 (Chaudhary et al., (1996) Plant Mol. Biol. 30:1247-57), rd29 (Kasuga et al., (1999) Nature Biotechnology 18:287-291); osmotic inducible promoters, such as Rab17 (Vilardell et al., (1991) Plant Mol. Biol. 17:985-93) and osmotin (Raghothama et al., (1993) Plant Mol Biol 23:1117-28); and heat inducible promoters, such as heat shock proteins (Barros et al., (1992) Plant Mol. 19:665-75; Marrs et al., (1993) Dev. Genet. 14:27-41), smHSP (Waters et al., (1996) J. Experimental Botany 47:325-338), and the heat-shock inducible element from the parsley ubiquitin promoter (WO 03/102198). Other stress-inducible promoters include rip2 (U.S. Pat. No. 5,332,808 and U.S. Publication No. 2003/0217393) and rd29a (Yamaguchi-Shinozaki et al., (1993) Mol. Gen. Genetics 236:331-340). Certain promoters are inducible by wounding, including the Agrobacterium pMAS promoter (Guevara-Garcia et al., (1993) Plant J. 4(3):495-505) and the Agrobacterium ORF13 promoter (Hansen et al., (1997) Mol. Gen. Genet. 254(3):337-343).

Tissue-preferred promoters can be utilized to target enhanced transcription and/or expression within a particular plant tissue. When referring to preferential expression, what is meant is expression at a higher level in the particular plant tissue than in other plant tissue. Examples of these types of promoters include seed preferred expression such as that provided by the phaseolin promoter (Bustos et al., (1989) The Plant Cell Vol. 1, 839-853), and the maize globulin-1 gene (Belanger, et al. (1991) Genetics 129:863-972). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, globulin 1, etc. Seed-preferred promoters also include those promoters that direct gene expression predominantly to specific tissues within the seed such as, for example, the endosperm-preferred promoter of γ-zein, the cryptic promoter from tobacco (Fobert et al., (1994) T-DNA tagging of a seed coat-specific cryptic promoter in tobacco. Plant J. 4: 567-577), the P-gene promoter from maize (Chopra et al., (1996) Alleles of the maize P gene with distinct tissue specificities encode Myb-homologous proteins with C-terminal replacements. Plant Cell 7:1149-1158, Erratum in Plant Cell. 1997, 1:109), the globulin-1 promoter from maize (Belenger and Kriz (1991) Molecular basis for Allelic Polymorphism of the maize Globulin-1 gene. Genetics 129: 863-972), and promoters that direct expression to the seed coat or hull of maize kernels, for example the pericarp-specific glutamine synthetase promoter (Muhitch et al., (2002) Isolation of a Promoter Sequence From the Glutamine Synthetase₁₋₂ Gene Capable of Conferring Tissue-Specific Gene Expression in Transgenic Maize. Plant Science 163:865-872).

In addition to the promoter, the gene expression cassette (which can be in, e.g., a vector) typically contains a transcription unit or expression cassette that contains all the additional elements required for the expression of the nucleic acid in host cells, either prokaryotic or eukaryotic. A typical expression cassette thus contains a promoter operably linked to a nucleic acid sequence encoding a gene product (e.g., a protein). The gene expression cassette may also include additional elements which are operably linked according to methods known art: signals required for efficient polyadenylation of the transcript, transcriptional termination, ribosome binding sites, or translation termination. Additionally, the expression cassette may include enhancers and/or heterologous splicing signals.

Other components of the gene expression cassette are provided as embodiments. Examples include selectable markers, targeting or regulatory sequences, transit peptide sequences such as the optimized transit peptide sequence (see U.S. Pat. No. 5,510,471) stabilizing sequences such as RB7 MAR (see Thompson and Myatt, (1997) Plant Mol. Biol., 34: 687-692 and International Patent Publication No. WO9727207) or leader sequences, introns etc. General descriptions and examples of plant expression vectors and reporter genes can be found in Gruber, et al., “Vectors for Plant Transformation” in Methods in Plant Molecular Biology and Biotechnology, Glick et al eds; CRC Press pp. 89-119 (1993). The selection of an appropriate expression vector will depend upon the host and the method of introducing the expression vector into the host. The gene expression cassette will also include at the 3′ terminus of the heterologous nucleotide sequence of interest, a transcriptional and translational termination region functional in plants. The termination region can be native with the promoter nucleotide sequence of embodiments of the present disclosure, can be native with the DNA sequence of interest, or can be derived from another source. Convenient termination regions are available from the Ti-plasmid of A. tumefaciens, such as the octopine synthase and nopaline synthase (nos) termination regions (Depicker et al., Mol. and Appl. Genet. 1:561-573 (1982) and Shaw et al. (1984) Nucleic Acids Research vol. 12, No. 20 pp7831-7846(nos)); see also Guerineau et al. Mol. Gen. Genet. 262:141-144 (1991); Proudfoot, Cell 64:671-674 (1991); Sanfacon et al. Genes Dev. 5:141-149 (1991); Mogen et al. Plant Cell 2:1261-1272 (1990); Munroe et al. Gene 91:151-158 (1990); Ballas et al., Nucleic Acids Res. 17:7891-7903 (1989); Joshi et al. Nucleic Acid Res. 15:9627-9639 (1987).

The gene expression cassettes can additionally contain 5′ leader sequences. Such leader sequences can act to enhance translation. Translation leaders are known in the art and include by way of example, picornavirus leaders, EMCV leader (Encephalomyocarditis 5′ noncoding region), Elroy-Stein et al., Proc. Nat. Acad. Sci. USA 86:6126-6130 (1989); potyvirus leaders, for example, TEV leader (Tobacco Etch Virus) Carrington and Freed Journal of Virology, 64:1590-1597 (1990), MDMV leader (Maize Dwarf Mosaic Virus), Allison et al., Virology 154:9-20 (1986); human immunoglobulin heavy-chain binding protein (BiP), Macejak et al., Nature 353:90-94 (1991); untranslated leader from the coat protein mRNA of alfalfa mosaic virus (AMV RNA 4), Jobling et al., Nature 325:622-625 (1987); Tobacco mosaic virus leader (TMV), Gallie et al., (1989) Molecular Biology of RNA, pages 237-256; and maize chlorotic mottle virus leader (MCMV) Lommel et al., Virology 81:382-385 (1991). See also Della-Cioppa et al., Plant Physiology 84:965-968 (1987).

The gene expression cassette construct can also contain sequences that enhance translation and/or mRNA stability such as introns. An example of one such intron is the first intron of gene II of the histone H3.III variant of Arabidopsis thaliana. Chaubet et al., Journal of Molecular Biology, 225:569-574 (1992).

In those instances where it is desirable for the expression cassette to express a gene product that is directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase and Helianthus annuus (U.S. Pat. No. 5,510,417), Zea mays Brittle-1 chloroplast transit peptide (Nelson et al., Plant Physiol 117(4):1235-1252 (1998); Sullivan et al., Plant Cell 3(12):1337-48; Sullivan et al., Planta (1995) 196(3):477-84; Sullivan et al., J. Biol. Chem. (1992) 267(26):18999-9004) and the like. In addition, chimeric chloroplast transit peptides are known in the art, such as the Optimized Transit Peptide (U.S. Pat. No. 5,510,471). Additional chloroplast transit peptides have been described previously in U.S. Pat. No. 5,717,084 and U.S. Pat. No. 5,728,925. One skilled in the art will readily appreciate the many options available in expressing a product to a particular organelle. For example, the barley alpha amylase sequence is often used to direct expression to the endoplasmic reticulum (Rogers, J. Biol. Chem. 260:3731-3738 (1985)).

It will be appreciated by one skilled in the art that use of recombinant DNA technologies can improve control of expression of transformed nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, stable integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno or Kozak sequences), modification of nucleic acid molecules to correspond to the codon usage of the host cell, and deletion of sequences that destabilize transcripts.

Reporter or marker genes for selection of transformed cells or tissues or plant parts or plants can be included in the transformation vectors. Examples of selectable markers include those that confer resistance to anti-metabolites such as herbicides or antibiotics, for example, dihydrofolate reductase, which confers resistance to methotrexate (Reiss, Plant Physiol. (Life Sci. Adv.) 13:143-149, 1994; see also Herrera Estrella et al., Nature 303:209-213, (1983); Meijer et al., Plant Mol. Biol. 16:807-820, (1991)); neomycin phosphotransferase, which confers resistance to the aminoglycosides neomycin, kanamycin and paromycin (Herrera-Estrella, EMBO J. 2:987-995, 1983 and Fraley et al., Proc. Natl. Acad. Sci USA 80:4803 (1983)) and hygromycin phosphotransferase, which confers resistance to hygromycin (Marsh, Gene 32:481-485, (1984); see also Waldron et al., Plant Mol. Biol. 5:103-108, (1985); Zhijian et al., Plant Science 108:219-227, (1995)); trpB, which allows cells to utilize indole in place of tryptophan; hisD, which allows cells to utilize histinol in place of histidine (Hartman, Proc. Natl. Acad. Sci., USA 85:8047, (1988)); mannose-6-phosphate isomerase which allows cells to utilize mannose (International Patent Application No. WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, (1995)).

Additional selectable markers include, for example, a mutant acetolactate synthase, which confers imidazolinone or sulfonylurea resistance (Lee et al., EMBO J. 7:1241-1248, (1988)), a mutant psbA, which confers resistance to atrazine (Smeda et al., Plant Physiol. 103:911-917, (1993)), or a mutant protoporphyrinogen oxidase (see U.S. Pat. No. 5,767,373), or other markers conferring resistance to an herbicide such as glufosinate. Examples of suitable selectable marker genes include, but are not limited to, genes encoding resistance to chloramphenicol (Herrera Estrella et al., EMBO J. 2:987-992, (1983)); streptomycin (Jones et al., Mol. Gen. Genet. 210:86-91, (1987)); spectinomycin (Bretagne-Sagnard et al., Transgenic Res. 5:131-137, (1996)); bleomycin (Hille et al., Plant Mol. Biol. 7:171-176, (1990)); sulfonamide (Guerineau et al., Plant Mol. Biol. 15:127-136, (1990)); bromoxynil (Stalker et al., Science 242:419-423, (1988)); glyphosate (Shaw et al., Science 233:478-481, (1986)); phosphinothricin (DeBlock et al., EMBO J. 6:2513-2518, (1987)), and the like.

One option for use of a selective gene is a glufosinate-resistance encoding DNA and in one embodiment can be the phosphinothricin acetyl transferase (pat), maize optimized pat gene or bar gene under the control of the Cassava Vein Mosaic Virus promoter. These genes confer resistance to bialaphos. See, (see, Wohlleben et al., (1988) Gene 70: 25-37); Gordon-Kamm et al., Plant Cell 2:603; 1990; Uchimiya et al., BioTechnology 11:835, 1993; White et al., Nucl. Acids Res. 18:1062, 1990; Spencer et al., Theor. Appl. Genet. 79:625-631, 1990; and Anzai et al., Mol. Gen. Gen. 219:492, 1989). A version of the pat gene is the maize optimized pat gene, described in U.S. Pat. No. 6,096,947.

In addition, markers that facilitate identification of a plant cell containing the polynucleotide encoding the marker may be employed. Scorable or screenable markers are useful, where presence of the sequence produces a measurable product and can produce the product without destruction of the plant cell. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al. The EMBO Journal vol. 6 No. 13 pp. 3901-3907); and alkaline phosphatase. In a preferred embodiment, the marker used is beta-carotene or provitamin A (Ye et al., Science 287:303-305-(2000)). The gene has been used to enhance the nutrition of rice, but in this instance it is employed instead as a screenable marker, and the presence of the gene linked to a gene of interest is detected by the golden color provided. Unlike the situation where the gene is used for its nutritional contribution to the plant, a smaller amount of the protein suffices for marking purposes. Other screenable markers include the anthocyanin/flavonoid genes in general (See discussion at Taylor and Briggs, The Plant Cell (1990)2:115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al., Plant Cell (1996) 8: 1171-1179; Scheffler et al., Mol. Gen. Genet. (1994) 242:40-48) and maize C2 (Wienand et al., Mol. Gen. Genet. (1986) 203:202-207); the B gene (Chandler et al., Plant Cell (1989) 1:1175-1183), the p1 gene (Grotewold et al., Proc. Natl. Acad. Sci USA (1991) 88:4587-4591; Grotewold et al., Cell (1994) 76:543-553; Sidorenko et al., Plant Mol. Biol. (1999)39:11-19); the bronze locus genes (Ralston et al., Genetics (1988) 119:185-197; Nash et al., Plant Cell (1990) 2(11): 1039-1049), among others.

Further examples of suitable markers include the cyan fluorescent protein (CYP) gene (Bolte et al., (2004) J. Cell Science 117: 943-54 and Kato et al., (2002) Plant Physiol 129: 913-42), the yellow fluorescent protein gene (PHIYFP™ from Evrogen; see Bolte et al., (2004) J. Cell Science 117: 943-54); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); a green fluorescent protein (GFP) gene (Sheen et al., Plant J. (1995) 8(5):777-84); and DsRed2 where plant cells transformed with the marker gene are red in color, and thus visually selectable (Dietrich et al., (2002) Biotechniques 2(2):286-293). Additional examples include a β-lactamase gene (Sutcliffe, Proc. Nat'l. Acad. Sci. U.S.A. (1978) 75:3737), which encodes an enzyme for which various chromogenic substrates are known (e.g., PADAC, a chromogenic cephalosporin); a xylE gene (Zukowsky et al., Proc. Nat'l. Acad. Sci. U.S.A. (1983) 80:1101), which encodes a catechol dioxygenase that can convert chromogenic catechols; an α-amylase gene (Ikuta et al., Biotech. (1990) 8:241); and a tyrosinase gene (Katz et al., J. Gen. Microbiol. (1983) 129:2703), which encodes an enzyme capable of oxidizing tyrosine to DOPA and dopaquinone, which in turn condenses to form the easily detectable compound melanin. Clearly, many such markers are available and known to one skilled in the art.

In certain embodiments, the nucleotide sequence of the transgene encoding a gene product in an expression cassette can be optionally combined with another nucleotide sequence of interest in the cassette and/or the plant. For example, in certain embodiments the transgene can be combined or “stacked” with another nucleotide sequence of interest that provides additional resistance or tolerance to glyphosate or another herbicide, and/or provides resistance to select insects or diseases and/or nutritional enhancements, and/or improved agronomic characteristics, and/or proteins or other products useful in feed, food, industrial, pharmaceutical or other uses. The “stacking” of two or more nucleic acid sequences of interest within a plant genome can be accomplished, for example, via conventional plant breeding using two or more events, transformation of a plant with a construct which contains the sequences of interest, re-transformation of a transgenic plant, or addition of new traits through integration via homologous recombination.

Such nucleotide sequences of interest include, but are not limited to, those examples of genes or coding sequences that confer (1) resistance to pests or disease, (2) resistance to herbicides, and (3) value added traits provided below:

1. Genes or Coding Sequences (e.g. iRNA) that Confer Resistance to Pests or Disease

(A) Plant Disease Resistance Genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. Examples of such genes include, the tomato Cf-9 gene for resistance to Cladosporium flavum (Jones et al., 1994 Science 266:789), tomato Pto gene, which encodes a protein kinase, for resistance to Pseudomonas syringae pv. tomato (Martin et al., 1993 Science 262:1432), and Arabidopsis RSSP2 gene for resistance to Pseudomonas syringae (Mindrinos et al., 1994 Cell 78:1089).

(B) A Bacillus thuringiensis protein, a derivative thereof or a synthetic polypeptide modeled thereon, such as, a nucleotide sequence of a Bt δ-endotoxin gene (Geiser et al., 1986 Gene 48:109), and a vegetative insecticidal (VIP) gene (see, e.g., Estruch et al., (1996) Proc. Natl. Acad. Sci. 93:5389-94). Moreover, DNA molecules encoding δ-endotoxin genes can be purchased from American Type Culture Collection (Rockville, Md.), under ATCC accession numbers 40098, 67136, 31995 and 31998.

(C) A lectin, such as, nucleotide sequences of several Clivia miniata mannose-binding lectin genes (Van Damme et al., 1994 Plant Molec. Biol. 24:825).

(D) A vitamin binding protein, such as avidin and avidin homologs which are useful as larvicides against insect pests. See U.S. Pat. No. 5,659,026.

(E) An enzyme inhibitor, e.g., a protease inhibitor or an amylase inhibitor. Examples of such genes include a rice cysteine proteinase inhibitor (Abe et al., 1987 J. Biol. Chem. 262:16793), a tobacco proteinase inhibitor I (Huub et al., 1993 Plant Molec. Biol. 21:985), and an α-amylase inhibitor (Sumitani et al., 1993 Biosci. Biotech. Biochem. 57:1243).

(F) An insect-specific hormone or pheromone such as an ecdysteroid and juvenile hormone a variant thereof, a mimetic based thereon, or an antagonist or agonist thereof, such as baculovirus expression of cloned juvenile hormone esterase, an inactivator of juvenile hormone (Hammock et al., 1990 Nature 344:458).

(G) An insect-specific peptide or neuropeptide which, upon expression, disrupts the physiology of the affected pest (J. Biol. Chem. 269:9). Examples of such genes include an insect diuretic hormone receptor (Regan, 1994), an allostatin identified in Diploptera punctata (Pratt, 1989), and insect-specific, paralytic neurotoxins (U.S. Pat. No. 5,266,361).

(H) An insect-specific venom produced in nature by a snake, a wasp, etc., such as a scorpion insectotoxic peptide (Pang, (1992) Gene 116:165).

(I) An enzyme responsible for a hyperaccumulation of monoterpene, a sesquiterpene, a steroid, hydroxamic acid, a phenylpropanoid derivative or another non-protein molecule with insecticidal activity.

(J) An enzyme involved in the modification, including the post-translational modification, of a biologically active molecule; for example, glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. Examples of such genes include, a callas gene (PCT published application WO93/02197), chitinase-encoding sequences (which can be obtained, for example, from the ATCC under accession numbers 3999637 and 67152), tobacco hookworm chitinase (Kramer et al., (1993) Insect Molec. Biol. 23:691), and parsley ubi4-2 polyubiquitin gene (Kawalleck et al., (1993) Plant Molec. Biol. 21:673).

(K) A molecule that stimulates signal transduction. Examples of such molecules include nucleotide sequences for mung bean calmodulin cDNA clones (Botella et al., (1994) Plant Molec. Biol. 24:757) and a nucleotide sequence of a maize calmodulin cDNA clone (Griess et al., (1994) Plant Physiol. 104:1467).

(L) A hydrophobic moment peptide. See U.S. Pat. Nos. 5,659,026 and 5,607,914; the latter teaches synthetic antimicrobial peptides that confer disease resistance.

(M) A membrane permease, a channel former or a channel blocker, such as a cecropin-β lytic peptide analog (Jaynes et al., (1993) Plant Sci. 89:43) which renders transgenic tobacco plants resistant to Pseudomonas solanacearum.

(N) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. See, for example, Beachy et al., (1990) Ann. Rev. Phytopathol. 28:451.

(O) An insect-specific antibody or an immunotoxin derived therefrom. Thus, an antibody targeted to a critical metabolic function in the insect gut would inactivate an affected enzyme, killing the insect. For example, Taylor et al., (1994) Abstract #497, Seventh Int'l. Symposium on Molecular Plant-Microbe Interactions shows enzymatic inactivation in transgenic tobacco via production of single-chain antibody fragments.

(P) A virus-specific antibody. See, for example, Tavladoraki et al., (1993) Nature 266:469, which shows that transgenic plants expressing recombinant antibody genes are protected from virus attack.

(Q) A developmental-arrestive protein produced in nature by a pathogen or a parasite. Thus, fungal endo α-1,4-D polygalacturonases facilitate fungal colonization and plant nutrient release by solubilizing plant cell wall homo-α-1,4-D-galacturonase (Lamb et al., (1992) Bio/Technology 10:1436). The cloning and characterization of a gene which encodes a bean endopolygalacturonase-inhibiting protein is described by (Toubart et al., (1992) Plant J. 2:367).

(R) A developmental-arrestive protein produced in nature by a plant, such as the barley ribosome-inactivating gene that provides an increased resistance to fungal disease (Longemann et al., (1992). Bio/Technology 10:3305).

(S) RNA interference, in which a DNA polynucleotide encoding an RNA molecule is used to inhibit expression of a target gene. An RNA molecule in one example is partially or fully double stranded, which triggers a silencing response, resulting in cleavage of dsRNA into small interfering RNAs, which are then incorporated into a targeting complex that destroys homologous mRNAs. See, e.g., Fire et al., U.S. Pat. No. 6,506,559; Graham et al., U.S. Pat. No. 6,573,099.

2. Genes or Coding Sequences that Confer Resistance to a Herbicide

(A) Genes encoding resistance or tolerance to a herbicide that inhibits the growing point or meristem, such as an imidazalinone, sulfonanilide or sulfonylurea herbicide. Exemplary genes in this category code for a mutant ALS enzyme (Lee et al., (1988) EMBOJ. 7:1241), which is also known as AHAS enzyme (Miki et al., (1990) Theor. Appl. Genet. 80:449).

(B) One or more additional genes encoding resistance or tolerance to glyphosate imparted by mutant EPSP synthase and aroA genes, or through metabolic inactivation by genes such as GAT (glyphosate acetyltransferase) or GOX (glyphosate oxidase) and other phosphono compounds such as glufosinate (pat and bar genes; DSM-2), and aryloxyphenoxypropionic acids and cyclohexanediones (ACCase inhibitor encoding genes). See, for example, U.S. Pat. No. 4,940,835, which discloses the nucleotide sequence of a form of EPSP which can confer glyphosate resistance. A DNA molecule encoding a mutant aroA gene can be obtained under ATCC Accession Number 39256, and the nucleotide sequence of the mutant gene is disclosed in U.S. Pat. No. 4,769,061. European Patent application No. 0 333 033 and U.S. Pat. No. 4,975,374 disclose nucleotide sequences of glutamine synthetase genes which confer resistance to herbicides such as L-phosphinothricin. The nucleotide sequence of a phosphinothricin acetyl-transferase gene is provided in European Patent application No. 0 242 246. De Greef et al., (1989) Bio/Technology 7:61 describes the production of transgenic plants that express chimeric bar genes coding for phosphinothricin acetyl transferase activity. Exemplary of genes conferring resistance to aryloxyphenoxypropionic acids and cyclohexanediones, such as sethoxydim and haloxyfop, are the Accl-S1, Accl-S2 and Accl-S3 genes described by Marshall et al., (1992) Theor. Appl. Genet. 83:435.

(C) Genes encoding resistance or tolerance to a herbicide that inhibits photosynthesis, such as a triazine (psbA and gs+ genes) and a benzonitrile (nitrilase gene). Przibilla et al., (1991) Plant Cell 3:169 describe the use of plasmids encoding mutant psbA genes to transform Chlamydomonas. Nucleotide sequences for nitrilase genes in U.S. Pat. No. 4,810,648, and DNA molecules containing these genes are available under ATCC accession numbers 53435, 67441 and 67442. Cloning and expression of DNA coding for a glutathione 5-transferase is described by Hayes et al., (1992) Biochem. J. 285:173.

(D) Genes encoding resistance or tolerance to a herbicide that bind to hydroxyphenylpyruvate dioxygenases (HPPD), enzymes which catalyze the reaction in which para-hydroxyphenylpyruvate (HPP) is transformed into homogentisate. This includes herbicides such as isoxazoles (European Patent No. 418175, European Patent No. 470856, European Patent No. 487352, European Patent No. 527036, European Patent No. 560482, European Patent No. 682659, U.S. Pat. No. 5,424,276), in particular isoxaflutole, which is a selective herbicide for maize, diketonitriles (European Patent No. 496630, and European Patent No. 496631), in particular 2-cyano-3-cyclopropyl-1-(2-502CH3-4-CF3 phenyl) propane-1,3-dione and 2-cyano-3-cyclopropyl-1-(2-SO2CH3-4-2,3Cl2phenyl) propane-1,3-dione, triketones (European Patent No. 625505, European Patent No. 625508, U.S. Pat. No. 5,506,195), in particular sulcotrione, and pyrazolinates. A gene that produces an overabundance of HPPD in plants can provide tolerance or resistance to such herbicides, including, for example, genes described in U.S. Pat. Nos. 6,268,549 and 6,245,968 and U.S. Patent Publication No. 20030066102.

(E) Genes encoding resistance or tolerance to phenoxy auxin herbicides, such as 2,4-dichlorophenoxyacetic acid (2,4-D) and which may also confer resistance or tolerance to aryloxyphenoxypropionate (AOPP) herbicides. Examples of such genes include the α-ketoglutarate-dependent dioxygenase enzyme (aad-1) gene, described in U.S. Pat. No. 7,838,733.

(F) Genes encoding resistance or tolerance to phenoxy auxin herbicides, such as 2,4-dichlorophenoxyacetic acid (2,4-D) and which may also confer resistance or tolerance to pyridyloxy auxin herbicides, such as fluroxypyr or triclopyr. Examples of such genes include the α-ketoglutarate-dependent dioxygenase enzyme gene (aad-12), described in WO 2007/053482 A2.

(G) Genes encoding resistance or tolerance to dicamba (see, e.g., U.S. Patent Publication No. 20030135879).

(H) Genes providing resistance or tolerance to herbicides that inhibit protoporphyrinogen oxidase (PPO) (see U.S. Pat. No. 5,767,373).

(I) Genes providing resistance or tolerance to triazine herbicides (such as atrazine) and urea derivatives (such as diuron) herbicides which bind to core proteins of photosystem II reaction centers (PS II) (See Brussian et al., (1989) EMBO J. 1989, 8(4): 1237-1245.

3. Genes That Confer or Contribute to a Value-Added Trait

(A) Modified fatty acid metabolism, for example, by transforming maize or Brassica with an antisense gene or stearoyl-ACP desaturase to increase stearic acid content of the plant (Knultzon et al., (1992) Proc. Nat. Acad. Sci. USA 89:2624.

(B) Decreased phytate content.

(1) Introduction of a phytase-encoding gene, such as the Aspergillus niger phytase gene (Van Hartingsveldt et al., (1993) Gene 127:87), enhances breakdown of phytate, adding more free phosphate to the transformed plant.

(2) A gene could be introduced that reduces phytate content. In maize, this, for example, could be accomplished by cloning and then reintroducing DNA associated with the single allele which is responsible for maize mutants characterized by low levels of phytic acid (Raboy et al., (1990) Maydica 35:383).

(C) Modified carbohydrate composition effected, for example, by transforming plants with a gene coding for an enzyme that alters the branching pattern of starch. Examples of such enzymes include, Streptococcus mucus fructosyltransferase gene (Shiroza et al., (1988) J. Bacteriol. 170:810), Bacillus subtilis levansucrase gene (Steinmetz et al., (1985) Mol. Gen. Genel. 200:220), Bacillus licheniformis α-amylase (Pen et al., (1992) Bio/Technology 10:292), tomato invertase genes (Elliot et al., (1993), barley amylase gene (Sogaard et al., (1993) J. Biol. Chem. 268:22480), and maize endosperm starch branching enzyme II (Fisher et al., (1993) Plant Physiol. 102:10450).

In further embodiments of the subject disclosure, the transgenic plant produces a commodity product. In an embodiment, the commodity product is selected from the group consisting of protein concentrate, protein isolate, grain, meal, flour, oil, or fiber.

A commodity product refers to any product which is comprised of material derived from a plant or plant seed and is sold to consumers. Crop plants are the largest source of protein, carbohydrates and vegetable oil for consumption. The transgenic plants can be used to manufacture commodity products. The plants and/or plant seeds can be processed into meal, flour, or oil as well as be used as a protein or oil source in animal feeds for both terrestrial and aquatic animals. Soybeans and soybean oils can be used in the manufacture of many different products, but not limited to, whole or processed seeds, animal feed, vegetable oil, meal, flour, nontoxic plastics, printing inks, lubricants, waxes, hydraulic fluids, electric transformer fluids, solvents, cosmetics, hair care products, natto, tempeh, protein concentrate, protein isolates, textured and hydrolyzed protein, and biodiesel.

In additional embodiments the subject disclosure relates to a transgenic plant, wherein the transgenic plant is selected from the group consisting of a dicotyledonous plant or a monocotyledonous plant. In further embodiments, the subject disclosure relates to consumable plants, including crop plants and plants used for their oils, protein, or carbohydrates. Thus, any plant species or plant cell can be selected as described further below.

In some embodiments, plants which are genetically modified in accordance with the present disclosure (e.g., plant host cells) includes, but is not limited to, any higher plants, including both dicotyledonous and monocotyledonous plants, and particularly consumable plants, including crop plants. Such plants can include, but are not limited to, for example: alfalfa, soybeans, cotton, rapeseed (also described as canola), linseed, corn, rice, brachiaria, wheat, safflowers, sorghum, sugarbeet, sunflowers, tobacco and turf grasses. Thus, any plant species or plant cell can be selected. In embodiments, plant cells used herein, and plants grown or derived therefrom, include, but are not limited to, cells obtainable from rapeseed (Brassica napus); indian mustard (Brassica juncea); Ethiopian mustard (Brassica carinata); turnip (Brassica rapa); cabbage (Brassica oleracea); soybean (Glycine max); linseed/flax (Linum usitatissimum); maize (also described as corn) (Zea mays); safflower (Carthamus tinctorius); sunflower (Helianthus annuus); tobacco (Nicotiana tabacum); Arabidopsis thaliana; Brazil nut (Betholettia excelsa); castor bean (Ricinus communis); coconut (Cocus nucifera); coriander (Coriandrum sativum); cotton (Gossypium spp.); groundnut (Arachis hypogaea); jojoba (Simmondsia chinensis); oil palm (Elaeis guineeis); olive (Olea eurpaea); rice (Oryza sativa); squash (Cucurbita maxima); barley (Hordeum vulgare); sugarcane (Saccharum officinarum); rice (Oryza sativa); wheat (Triticum spp. including Triticum durum and Triticum aestivum); and duckweed (Lemnaceae sp.). In some embodiments, the genetic background within a plant species may vary.

The nucleic acids introduced into a plant cell can be used to confer desired traits on essentially any plant. A wide variety of plants and plant cell systems may be engineered for the desired physiological and agronomic characteristics described herein using the gene expression constructs of the present disclosure and the various transformation methods mentioned above. In embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Thus, the disclosed methods and compositions have use over a broad range of plants, including, but not limited to, species from the genera Asparagus, Avena, Brassica, Citrus, Citrullus, Capsicum, Cucurbita, Daucus, Erigeron, Glycine, Gossypium, Hordeum, Lactuca, Lolium, Lycopersicon, Malus, Manihot, Nicotiana, Orychophragmus, Oryza, Persea, Phaseolus, Pisum, Pyrus, Prunus, Raphanus, Secale, Solanum, Sorghum, Triticum, Vitis, Vigna, and Zea mays.

A transformed plant cell, callus, tissue or plant may be identified and isolated by selecting or screening the engineered plant material for traits encoded by the marker genes present on the transforming DNA. For instance, selection can be performed by growing the engineered plant material on media containing an inhibitory amount of the antibiotic or herbicide to which the transforming gene construct confers resistance. Further, transformed plants and plant cells can also be identified by screening for the activities of any visible marker genes (e.g., the β-glucuronidase, luciferase, or gfp genes) that may be present on the recombinant nucleic acid constructs. Such selection and screening methodologies are well known to those skilled in the art. Molecular confirmation methods that can be used to identify transgenic plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay, otherwise known as TAQMAN® (Life Technologies, Foster City, Calif.), is a method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. Such a hydrolysis probe assay for detection of as an amplification reaction is an embodiment of the subject disclosure.

KASPar® assays are a method of detecting and quantifying the presence of a DNA sequence. Briefly, the genomic DNA sample comprising the integrated gene expression cassette polynucleotide is screened using a polymerase chain reaction (PCR) based assay known as a KASPar® assay system. The KASPar® assay used in the practice of the subject disclosure can utilize a KASPar® PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the KASPar® PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence. Such a KASPar® assay for detection of an amplification reaction is an embodiment of the subject disclosure.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed, such as YO-PRO-1®, SYTOX Green®, SYBR Green I®, SYTO11®, SYTO12®, SYTO13®, BOBO®, YOYO®, and TOTO®. in one embodiment, a second fluorescent DNA dye is SYTO13® used at less than 10 μM, less than 4 μM, or less than 2.7 μM.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction. Three NGS platforms are commercially available, the Genome Sequencer FLX™ from 454 Life Sciences/Roche, the Illumina Genome Analyser™ from Solexa and Applied Biosystems' SOLiD™ (acronym for: ‘Sequencing by Oligo Ligation and Detection’). In addition, there are two single molecule sequencing methods that are currently being developed. These include the true Single Molecule Sequencing (tSMS) from Helicos Bioscience™ and the Single Molecule Real Time™ sequencing (SMRT) from Pacific Biosciences.

The Genome Sequencher FLX™ which is marketed by 454 Life Sciences/Roche is a long read NGS, which uses emulsion PCR and pyrosequencing to generare sequencing reads. DNA fragments of 300-800 bp or libraries containing fragments of 3-20 kbp can be used. The reactions can produce over a million reads of about 250 to 400 bases per run for a total yield of 250 to 400 megabases. This technology produces the longest reads but the total sequence output per run is low compared to other NGS technologies.

The Illumina Genome Analyser™ which is marketed by Solexa™ is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The Sequencing by Oligo Ligation and Detection (SOLiD) system marketed by Applied Biosystems™ is a short read technology. This NGS technology uses fragmented double stranded DNA that are up to 10 kbp in length. The system uses sequencing by ligation of dye-labelled oligonucleotide primers and emulsion PCR to generate one billion short reads that result in a total sequence output of up to 30 gigabases per run.

tSMS of Helicos Bioscience™ and SMRT of Pacific Biosciences™ apply a different approach which uses single DNA molecules for the sequence reactions. The tSMS Helicos™ system produces up to 800 million short reads that result in 21 gigabases per run. These reactions are completed using fluorescent dye-labelled virtual terminator nucleotides that is described as a ‘sequencing by synthesis’ approach.

The SMRT Next Generation Sequencing system marketed by Pacific Biosciences™ uses a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

In another embodiment, the detection can be completed using blotting assays, including Western blots, Northern blots, and Southern blots. Such blotting assays are commonly used techniques in biological research for the identification and quantification of biological samples. These assays include first separating the sample components in gels by electrophoretic means, followed by transfer of the electrophoretically separated components from the gels to transfer membranes that are made of materials such as nitrocellulose, polyvinylidene fluoride (PVDF), or Nylon. Analytes can also be directly spotted on these supports or directed to specific regions on the supports by applying vacuum, capillary action, or pressure, without prior separation. The transfer membranes are then commonly subjected to a post-transfer treatment to enhance the ability of the analytes to be distinguished from each other and detected, either visually or by automated readers.

In a further embodiment the detection can be completed using an ELISA assay, which uses a solid-phase enzyme immunoassay to detect the presence of a substance, usually an antigen, in a liquid sample or wet sample. Antigens from the sample are attached to a surface of a plate. Then, a further specific antibody is applied over the surface so it can bind to the antigen. This antibody is linked to an enzyme, and, in the final step, a substance containing the enzyme's substrate is added. The subsequent reaction produces a detectable signal, most commonly a color change in the substrate.

In other embodiments, the gene expression cassette may be introduced in the context of inserting a nucleic acid into the genome of a cell, including transformation into the cell, as well as crossing a plant having the sequence with another plant, so that the second plant contains the heterologous sequence, as in conventional plant breeding techniques. Such breeding techniques are well known to one skilled in the art. For a discussion of plant breeding techniques, see Poehlman (1995) Breeding Field Crops, AVI Publication Co., Westport Conn, 4^{th} Edit. Backcrossing methods may be used to introduce a gene into the plants. This technique has been used for decades to introduce traits into a plant. An example of a description of this and other plant breeding methodologies that are well known can be found in references such as Poehlman, supra, and Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

Certain embodiments relate to processes of making crosses using a plant of an embodiment of this disclosure as at least one parent. For example, particular embodiments relate to an F₁ hybrid plant having as one or both parents any of the plants exemplified herein. Other embodiments relate to seed produced by such F₁ hybrids. Still other embodiments relate to a method for producing an F₁ hybrid seed by crossing an exemplified plant with a different (e.g. in-bred parent) plant and harvesting the resultant hybrid seed. Other embodiments relate to an exemplified plant that is either a female parent or a male parent. Characteristics of the resulting plants may be improved by careful consideration of the parent plants.

A transgenic plant of an embodiment of the subject disclosure can be bred by first sexually crossing a first parental plant consisting of a plant grown from seed of any one of the lines referred to herein, and a second parental plant, thereby producing a plurality of first progeny plants; then selecting a first progeny plant that is resistant to glyphosate; selfing the first progeny plant, thereby producing a plurality of second progeny plants; and then selecting from the second progeny plants a plant that is resistant to glyphosate. These steps can further include the back-crossing of the first progeny plant or the second progeny plant to the second parental plant or a third parental plant. A crop comprising seeds of particular embodiments, or progeny thereof, can then be planted.

It is also to be understood that two different transgenic plants can also be mated to produce offspring that contain two independently segregating, added, exogenous genes. Selfing of appropriate progeny can produce plants that are homozygous for both added, exogenous genes. Backcrossing to a parental plant and out-crossing with a non-transgenic plant are also contemplated, as is vegetative propagation. Other breeding methods commonly used for different traits and crops are known in the art. Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting.

### EXAMPLES

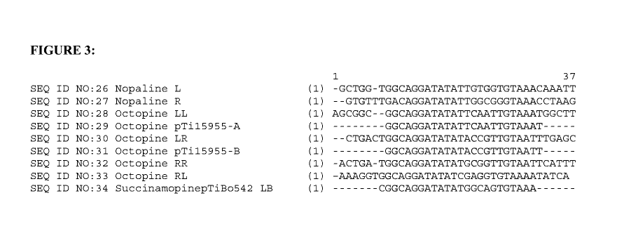

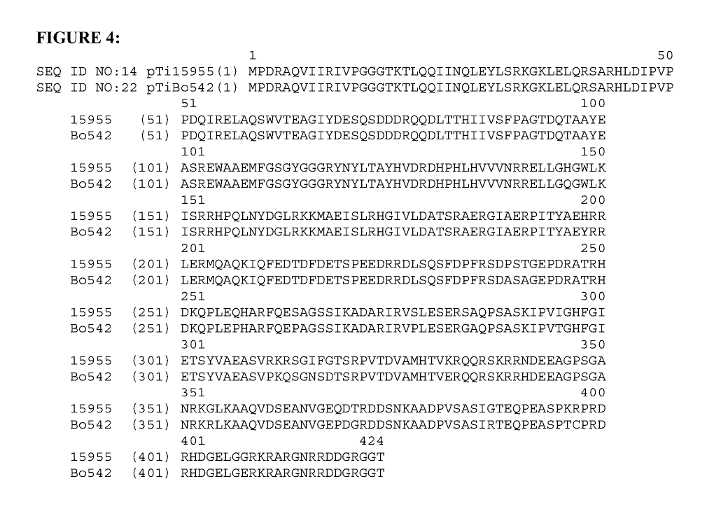

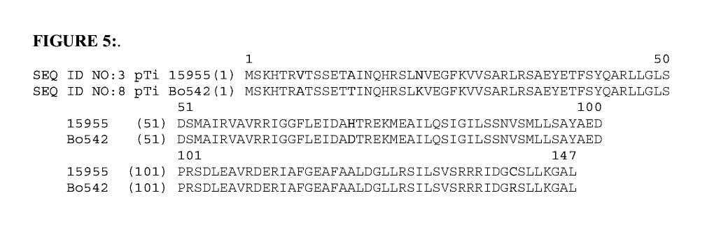

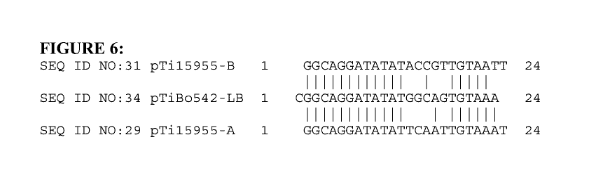

Divergent strains of Agrobacterium are known and have been reported in the literature as described previously. Typically, the strains of Agrobacterium are categorized as octopine, nopaline, or null (also called mannopine) synthesizing type strains. The various strains differ in the type of pTi plasmid that they contain. Included on the pTi plasmids (e.g., pTiBo542, pTiC58 [and the common derivative pTi15955], pTiAch5, or a pTiChry5), amongst other gene features, are octopine synthesizing genes, oncogenes, virulent genes (herein after vir genes), and inverted repeated T-DNA border sequences which flank the T-strand. The vir genes of the different types of Agrobacterium strains share high to moderate levels of sequence similarity. For example, the VirD2 protein sequences derived from the pTi helper plasmids of pTiBo542 and pTi15955 share 94% sequence identity (FIG. 4). Likewise the VirD1 protein sequences derived from the pTi helper plasmids of pTiBo542 and pTi15955 share 96.6% sequence identity (FIG. 5). Other features from the pTi plasmid share moderate levels of sequence identity. For example, the border sequences from pTi15955 and pTiBo542 share 75-80% sequence identity (FIG. 6). Despite the divergence in sequence identity, the VirD1 and VirD2 proteins from pTi15955 and pTiBo542 pTi plasmids were thought to bind and cleave to border sequences from pTi15955 and pTiBo542 pTi plasmids with equal levels of affinity.

The virD1 and virD2 gene sequences were obtained from pTi15955 (originating from an octopine strain), and incorporated into a plasmid within a strain of Agrobacterium that previously contained only the virD1 and virD2 gene sequences from pTiBo542 (originating from a succinamopine strain). The expression of the additional VirD1 and VirD2 proteins originating from an octopine strain of Agrobacterium were tested to determine if the existing border sequences that originated from an octopine strain of Agrobacterium would be cleaved with a higher level of efficiency. As such, the addition of the VirD1 and VirD2 proteins originating from the octopine strain of Agrobacterium would correct any incompatibilities (e.g., VirD1 and VirD2 proteins from a succinamopine strain of Agrobacterium, in combination with border sequences from an octopine strain of Agrobacterium) resulting in inefficient binding and cleavage of the border sequences.

The complementation was tested in Agrobacterium tumefaciens strain DA2552, in which a DNA fragment derived from obtained from pTi15955 and containing virD1 and virD2 along with the native VirD promoter (SEQ ID NO:35) was cloned into the resident booster (i.e. ternary) plasmid pDAB9292 which includes a 14.8 Kpn1 VirBCDG fragment isolated from pSB1 as previously described in International Patent Application No. 2012/016222 A2. This modified strain, also contained a binary expression vector which expressed the yfp and aad-1 transgenes as gene expression cassettes, and the pTiBo542 plasmid. The strain was used to transform Zea mays c.v. B104 immature embryos, and data for transformation frequency and molecular analyses were collected and analyzed.

### Example 1

### Amplification of VirD Promoter, VirD1 and VirD2 Genes from Agrobacterium

A PCR reaction was completed to amplify the VirD promoter, virD1 and virD2 sequence as a single fragment from the Agrobacterium pTi15955 plasmid. Primers were designed to incorporate SpeI restriction sites in the amplified product to facilitate cloning. Primer sequences were as follows: VirD-SpeI-F (SEQ ID NO:36) AAACTAGTGGGATCAGAAGCAGGTTTGA and VirD-SpeI-R (SEQ ID NO:37) AAACTAGTTGTCTCTAGGTCCCCCCG. PCR was set up using the Phusion Hot Start Flex 2× Master Mix® (New England Biolabs) and 0.5 μM of each primer. The PCR conditions were set at 98° C. for 10 min., followed by 35 cycles of 98° C. for 10 sec. and 72° C. for 30 sec., and a final extension at 72° C. for 7 min. The amplified PCR products were electrophoresed on a 0.8% agarose gel and the resulting ˜2400 bp band was gel-eluted using the Qiagen Gel Extraction Kit™ following the manufacturer's protocol. The DNA fragment was cloned into the pCR-Blunt II TOPO™ vector (Invitrogen) to create a plasmid construct that was validated by sequencing the inserted DNA.

### Example 2

### Cloning of VirD Promoter, virD1 and virD2 Genes into the Ternary Booster Plasmid

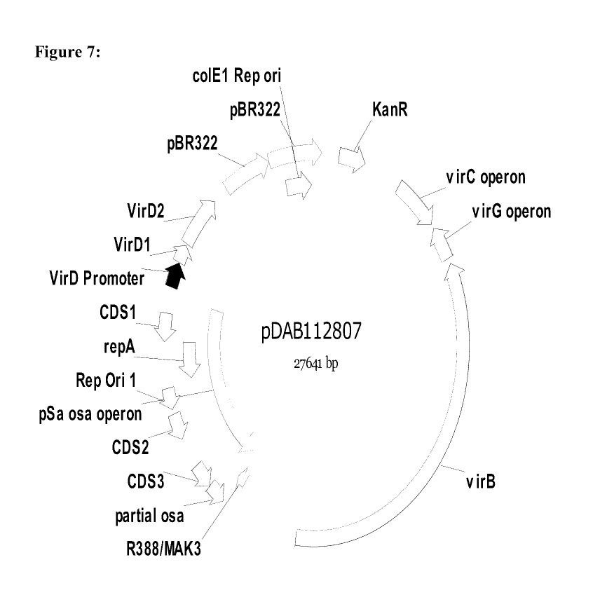

The PCR amplicon containing the virD1 and virD2 coding sequences was cloned into the pDAB9292 plasmid as a SpeI fragment, and transformed into Escherichia coli Stb13 chemically competent cells. Bacterial colonies were verified by colony PCR using a sense primer set located in pDAB9292 upstream of the SpeI site (pDAB9292-Spe-F (SEQ ID NO:38): GGCCGCGCAGCCACC) and an antisense primer in the VirD promoter (VirD-R1 (SEQ ID NO:39): CAAATGCACTCCGTTTCACAGGACAG). The new plasmid construct was designated as pDAB112807 (FIG. 7). The pDAB112807 construct was confirmed via digestion with restriction enzymes; PstI, SalI, and BglII on an agarose gel.

### Example 3

### Transformation of Agrobacterium Strain DA2552 with pDAB112807

Electrocompetent cells of Agrobacterium strain DA2552 were transformed with pDAB112807, and transformed cells were plated on YEP agar plates with appropriate antibiotics. The plates were incubated at 28° C. for two days. Ten individual colonies were isolated and streaked on a patch plate of YEP agar supplemented with appropriate antibiotics, and onto a patch plate of lactose agar. Both plates were incubated overnight at 28° C. The colonies streaked onto the lactose agar were tested via the ketolactose test. One colony that grew up on both plates, and produced a positive result with the ketolactose test was cultured overnight and stored as a sterile glycerol stock. Plasmid minipreps were isolated from the culture and digested with restriction enzymes; PstI, SalI, BglII and SphI on the capillary electrophoresis on an AdvanCE Capillary Electrophoresis System™. The novel Agrobacterium strain that contained pDAB112807 was labeled as Agrobacterium strain NS-1.

### Example 4

### Transformation of Agrobacterium Strain NS-1 with pDAB101556

Agrobacterium strain NS-lwas prepared for electroporation, and was transformed with pDAB101556 (a binary expression vector containing the yfp reporter and aad-1 herbicide selectable markers). Colonies containing pDAB101556 were isolated, and plasmid minipreps were isolated from the culture and digested with restriction enzymes; SalI, PstI, HindIII and SphI on the AdvanCE Capillary Electrophoresis System™.

### Example 5

### Transformation of Zea mays c.v.B104 Immature Embryos with Agrobacterium Culture Initiation

Glycerol stocks of Agrobacterium tumefaciens strain NS-1 (VirD modified ternary), and a control strain of Agrobacterium strain DA2552 (regular ternary strain) containing the pDAB101556 binary plasmid were obtained. The Agrobacterium cultures were streaked from glycerol stocks onto AB minimal medium, and incubated at 20° C. in the dark for 3 days. The cultures were then re-streaked onto a plate of YEP medium, and incubated at 20° C. in the dark for 1 day.

On the first day of an experiment, a mixture of Inoculation medium and acetosyringone were prepared in a volume appropriate to the number of constructs in the experiment. Inoculation medium was pipetted into a sterile, disposable, 250 ml flask. A 1 M stock solution of acetosyringone in 100% dimethyl sulfoxide was added to the flask containing inoculation medium in a volume appropriate to make a final acetosyringone concentration of 200 μM.

For each strain, 1-2 loops of Agrobacterium from the YEP plate were suspended in 15 ml of the inoculation medium/acetosyringone mixture inside a sterile, disposable, 50 ml centrifuge tube, and the optical density of the solution at 600 nm (O.D.₆₀₀) was measured using a spectrophotometer. The suspension was then diluted down to 0.25-0.35 O.D.₆₀₀ using additional Inoculation medium/acetosyringone mixture. The tube of Agrobacterium suspension was then placed horizontally on a platform shaker set at about 75 rpm at room temperature for between 1 and 4 hours before use.

### Ear Sterilization and Embryo Isolation

Ears from Zea mays c.v. B104 were harvested 10-12 days post pollination. Harvested ears were de-husked and surface-sterilized by immersion in a 20% solution of commercial bleach (Ultra Clorox® germicidal bleach, 6.15% sodium hypochlorite) and two drops of Tween-20™, for 20 minutes, followed by three rinses in sterile, deionized water inside a laminar flow hood. Immature zygotic embryos (1.8-2.2 mm long) were aseptically excised from each ear and distributed into one or more micro-centrifuge tubes containing 2.0 ml of Agrobacterium suspension into which 2 μl of 10% Break-Thru® 5233 surfactant had been added. The numbers of embryos used in each replicated experiment are given in Table 1.

TABLE 1
Number of Zea mays c.v. B104 immature embryos used in each replicate
with the regular ternary (Reg Ter) and modified ternary to include
additional copies of virD1 and virD2 (VirD) Agrobacterium strains.

| Embryos | Reg Ter | VirD |
|---|---|---|
| Rep 1 | 150 | 147 |
| Rep 2 | 151 | 150 |
| Rep 3 | 200 | 200 |
| Rep 4 | 158 | 166 |
| Rep 5 | 150 | 146 |
| Rep 6 | 177 | — |

### Agrobacterium Co-Cultivation

Upon completion of the embryo isolation activity the tube of embryos was closed and placed on a rocker platform for 5 minutes. The contents of the tube were then poured out onto a plate of co-cultivation medium, and the liquid Agrobacterium suspension was removed with a sterile, disposable, transfer pipette. The embryos were oriented with the scutellum facing up. The plate was then closed, sealed with 3M Micropore Tape™, and placed in an incubator at 25° C. with 24 hours/day light at approximately 60 μmol m⁻² s⁻¹ light intensity.

### Callus Selection and Regeneration of Transgenic Events

Following the co-cultivation period, embryos were transferred to Resting medium. No more than 36 embryos were moved to each plate. The plates were wrapped with 3M micropore Tape™ and incubated at 27° C. with 24 hours/day light at approximately 50 μmol m⁻¹ s⁻¹ light intensity for 7-10 days. Callused embryos were then transferred onto Selection I medium. No more than 18 callused embryos were moved to each plate of Selection I. The plates were wrapped with 3M micropore Tape™ and incubated at 27° C. with 24 hours/day light at approximately 50 μmol m⁻² s⁻¹ light intensity for 7 days. Callused embryos were then transferred to Selection II medium. No more than 12 callused embryos were moved to each plate of Selection II. The plates were wrapped with 3M micropore Tape™ and incubated at 27° C. with 24 hours/day light at approximately 50 μmol m⁻² s⁻¹ light intensity for 14 days.

At this stage, resistant calli were moved to Pre-Regeneration medium. No more than 9 calli were moved to each plate of Pre-Regeneration medium. The plates were wrapped with 3M micropore Tape™ and incubated at 27° C. with 24 hours/day light at approximately 50 μmol m⁻² s⁻¹ light intensity for 7 days. Regenerating calli were then transferred to Regeneration medium in Phytatrays™, and incubated at 28° C. with 16 hours light/8 hours dark per day at approximately 150 μmol m⁻² s⁻¹ light intensity for 14 days or until shoots developed. No more than 7 calli were placed in each Phytatray™. Leaf tissue was then collected for molecular analysis.

### Example 6

### Molecular Analysis of Transgenic Plants

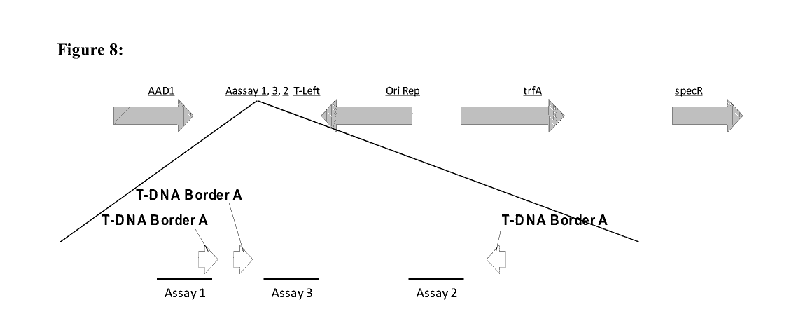

Molecular analyses were carried out on a total of 343 tissue samples collected for both treatments from 6 biological replicates. Real-time quantitative PCR (qPCR) were completed to assess insert copy number based on amplification of the aad-1 gene. Then a series of qPCR reactions were completed to evaluate the presence of the yfp gene. To assess the presence of vector backbone in the transgenic plants, a comprehensive set of qPCR assays were developed that covered the triple T-DNA border A, origin of replication, trfA and the spectinomycin gene (FIG. 8). A List of primers and probes used in the assays are provided in Table 2. Differences in means for all data were statistically computed following the Tukey-Kramer analysis method using JMP Pro 10™.

TABLE 2
Sequences of primers and probes used for aad-1, yfp, Agrobacterium
contamination, and vector backbone detection assays.

| | | SEQ ID | | |
|---|---|---|---|---|
| Name | Length | NO: | Sequence | Description |
| AAD1_F | 20 | 40 | TGTTCGGTTCCCTCTACCAA | AAD1 |
| AAD1_R | 22 | 41 | CAACATCCATCACCTTGACTGA | |
| AAD1_Probe | 24 | 42 | CACAGAACCGTCGCTTCAGCAACA | |
| BorderA_F11 | 25 | 43 | GCCAATTTTAGACAAGTATCAAACG | Border A1 |
| BorderA_R11 | 24 | 44 | GGCAGGATATATTCAATTGTAAAT | |
| Border1_1Probe | 23 | 45 | ATTGACGCTTAGACAACTTAATA | |
| BorderA_F2 | 27 | 46 | CTCTTTCTTTTTCTCCATATTGACC | Border A2 |
| | | | AT | |
| BorderA_R21 | 19 | 47 | GGCTTCATGTCCGGGAAAT | |
| BorderA_2Probe | 19 | 48 | ATACTCATTGCTGATCCAT | |
| BorderA_F3 | 19 | 49 | CGCGTGTCGAGGAATTCTG | Border A3 |
| BorderA_R31 | 26 | 50 | TCTTTATTTCGACGTGTCTACATTC | |
| | | | A | |
| BorderA_3Probe | 14 | 51 | CTGGCCCCCATTTG | |
| T-Left_F | 21 | 52 | GATGGCGTTGCGAGAGAAGCA | 172 bp |
| T-Left_R | 21 | 53 | GGCTCGATTGTACCTGCGTTC | downstream of |
| T-Left_Probe | 24 | 54 | TCCGTGAGATCAGCCGACGCACCG | BorderA |
| Ori-Rep_F | 21 | 55 | CGAGCGGGTGTTCCTTCTTCA | Ori Rep |
| Ori-Rep_R | 22 | 56 | CATCCGCTTGCCCTCATCTGTT | |
| Ori-Rep_Probe | 26 | 57 | TGCTCAACGGGAATCCTGCTCTGC | |
| | | | GA | |
| TRF_F | 17 | 58 | CCCGCTCAAGCTGGAAA | trfA |
| TRF_R | 18 | 59 | CAATGCACCAGGTCATCA | |
| TRF_Probe | 15 | 60 | CGGCGAAGCCTGCGA | |
| Spec_F | 19 | 61 | GACCGTAAGGCTTGATGAA | SpecR |
| Spec_Probe | 21 | 62 | CGAGATTCTCCGCGCTGTAGA | |
| Spec_R | 19 | 63 | CTTAGCTGGATAACGCCAC | |
| PicA_F | 26 | 64 | CAATCGAAATATGACGAAATGGA | Agrobacterium |
| | | | AA | contamination |
| PicA_R | 18 | 65 | GCATGATCCTTCGGCTG | detection |
| PicA_Probe | 17 | 66 | ACGACATCCGCCTGCT | |
| YFP125F | 19 | 67 | GATGCCTCAGTGGGAAAGG | YFPv2 |
| YFP125R | 23 | 68 | CCATAGGTGAGAGTGGTGACAA | |
| UPL125 | 8 | 69 | CTTGGAGC | |
| INV_F | 18 | 70 | TGGCGGACGACGACTTGT | Invertase |
| INV_R | 19 | 71 | AAAGTTTGGAGGCTGCCGT | Endogenous |
| INV_Probe | 21 | 72 | CGAGCAGACCGCCGTGTACTT | Reference |

### Example 7

### Transformation Frequency

Zea mays embryos infected with the different strains of Agrobacterium described above were regenerated on tissue culture media. The number of transgenic events regenerated for each transformation campaign were recorded and leaf tissue was collected for molecular analyses. Results for percentage of transformation frequency (% TF) and vector backbone integration (% BB) were calculated for events regenerated for 6 replicates using the regular ternary strain (Reg Ternary) and 5 replicates using the ternary strain modified to include additional virD sequences (VirD) as shown in Table 3.

TABLE 3
Percentage of transformation frequency (TF) and
transgenic plants with vector backbone (BB) in all replicates.

| | REP | % TF | % BB | Events Analyzed |
|---|---|---|---|---|
| Reg Ternary | 1 | 13.3 | 28.6 | 21 |
| Reg Ternary | 2 | 14.7 | 45.5 | 22 |
| Reg Ternary | 3 | 10.7 | 26.3 | 19 |
| Reg Ternary | 4 | 32.9 | 24.5 | 48 |
| Reg Ternary | 5 | 41.1 | 33.9 | 62 |
| Reg Ternary | 6 | 49.0 | 50.0 | 98 |
| VirD | 1 | 10.8 | 16.7 | 18 |
| VirD | 2 | 7.5 | 0.0 | 11 |
| VirD | 3 | 18.0 | 12.5 | 39 |
| VirD | 4 | 27.4 | 25.9 | 27 |
| VirD | 5 | 34.5 | 14.5 | 69 |

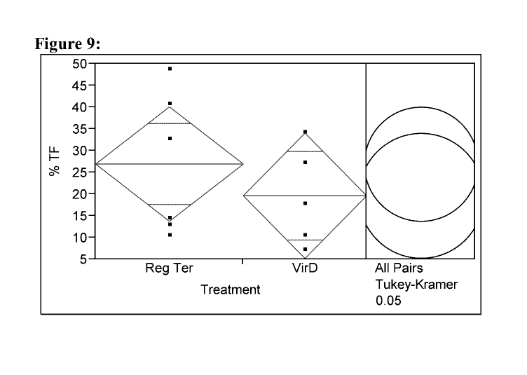

Transformation frequency did not vary significantly between the two strains (FIG. 9; Table 4). However, due to the inclusion of Haloxyfop Methyl Ester, instead of R-Haloxyfop Acid, in the selection media, overall transformation frequencies were greatly reduced in the regular ternary strain (Reg Ternary) experiments for replicates 4-6, ternary strain modified to include additional virD sequences (VirD) for replicates 4-5.

TABLE 4
Percentage of transformation frequency (TF), transgenic plants with vector
backbone (BB) and simple or multi-copy events. P-values for
significant differences (P < 0.05) are identified with an asterisk (i.e., *).

| | | | | | % | Events |
|---|---|---|---|---|---|---|
| | % TF | % BB | % 1-copy | % 2-copy | Multi-copy | Analyzed |
| Reg Ternary | 26.9 ± 6.6 | 34.8 ± 4.3 | 50.6 ± 1.7 | 22.6 ± 2.4 | 25.1 ± 2.4 | 270 |
| VirD | 19.6 ± 5.0 | 13.9 ± 4.2 | 58.1 ± 2.7 | 16.4 ± 4.5 | 22.5 ± 5.9 | 168 |
| p-Value | 0.419 | 0.007* | 0.036* | 0.228 | 0.668 | — |

### Vector Backbone in Transgenic Events

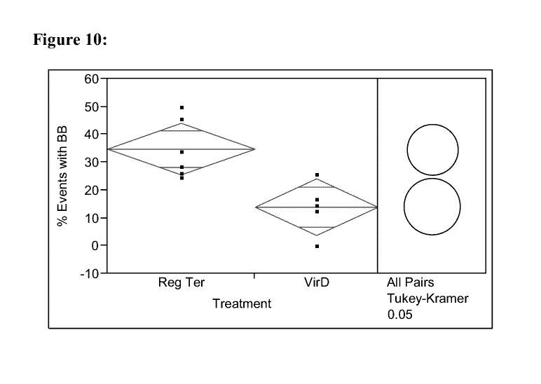

Molecular analyses were carried out to detect the presence of vector backbone (BB) sequences in the regenerated transgenic events. TaqMan™ PCR assays were designed to detect sequences in the T-left, OriRep, trfA and spectinomycin regions of pDAB101556. Around 35% of events generated using the regular ternary Agrobacterium strain showed the presence of backbone sequences. Comparatively, only around 14% of events generated using the modified VirD ternary strain showed the presence of backbone sequences (FIG. 10; Table 4). Thus, expression of the octopine VirD2 proteins in combination with T-DNA borders that originated from an octopine strain significantly decreased the occurrence of vector backbone integration in the transgenic plants.

### Transgene Copy Number in Transgenic Events

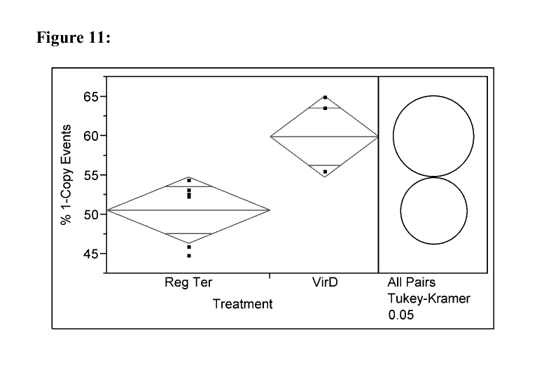

Transgene copy number was assessed by quantitative TaqMan™ PCR assay for aad-1. There were significantly higher number of single-copy events generated in samples treated with the VirD modified ternary strain as compared to those infected with the regular ternary strain (FIG. 11; Table 4).

Multi-copy events often contain multiple transgenes that are integrated as tandem repeats of the entire binary plasmid as a result of continuous read-through of the T-strands due to incomplete or inefficient processing of the Border A. Thus, the activity of the octopine VirD1 and VirD2 protein in recognizing and nicking the borders efficiently results in not only producing vector backbone-free T-strands, but also decreases the possibility of tandem repeats, leading to increase in single-copy events.

Addition of the octopine virD1 and virD2 sequences into Agrobacterium strains containing the disarmed pTiBo542 resulted in significantly decreased frequency of vector backbone in the transgenic plants along with a significantly increased frequency of 1-copy events. These results are consistent with octopine-derived VirD1 and VirD2 proteins recognizing the octopine-derived T-DNA borders of the binary plasmid with greater efficiency, resulting in proper processing of the T-strand.

The foregoing demonstrates various examples of the disclosed system for Agrobacterium-mediated transformation shows that the novel system results in an efficient transformation method.

What is claimed is:

1. A system for Agrobacterium-mediated transformation of a plant comprising:
a. a first plasmid comprising a first virD1 coding sequence and a first virD2 coding sequence, wherein the first virD1 coding sequence and the first virD2 coding sequence originate from a bacterial strain that synthesizes octopine;
b. a second plasmid comprising an Agrobacterium T-DNA border, wherein the Agrobacterium T-DNA border originates from a bacterial strain that synthesizes octopine; and,
c. a third plasmid comprising a second virD1 coding sequence and a second virD2 coding sequence, wherein the second virD1 coding sequence and the second virD2 coding sequence originate from a bacterial strain that synthesizes succinamopine.

2. The system of claim 1, further comprising a T-DNA region operably linked to the Agrobacterium T-DNA border.

3. The system of claim 2, wherein the T-DNA region comprises a gene expression cassette.

4. The system of claim 3, wherein the gene expression cassette comprises a selectable marker.

5. The system of claim 2, wherein said second plasmid further comprises a second Agrobacterium T-DNA border, wherein said T-DNA border is operably linked to one end of the T-DNA region, and said second T-DNA border is operably linked to the other end of the T-DNA region.

6. The system of claim 5, wherein the first and second T-DNA borders are independently selected from the group consisting of a nopaline Agrobacterium T-DNA border, an octopine Agrobacterium T-DNA border, a succinamopine Agrobacterium T-DNA border, or any combination thereof.

7. The system of claim 5, wherein the first and second T-DNA borders are T-DNA border sequences originating from an octopine synthesizing Agrobacterium.

8. The system of claim 7, wherein the first plasmid comprises a third virD1 coding sequence, wherein the third virD1 coding sequence originates from a bacterial strain that synthesizes succinamopine.

9. The system of claim 1, wherein the second plasmid comprises the second virD1 coding sequence, wherein the second virD1 coding sequence originates from a bacterial strain that synthesizes octopine.

10. The system of claim 1, wherein the second plasmid comprises the second virD2 coding sequence, wherein the second virD2 coding sequence originates from a bacterial strain that synthesizes octopine.

11. The system of claim 1, further comprising a VirBCDG fragment integrated within the first plasmid, second plasmid, third plasmid, or an Agrobacterium genomic DNA.

12. An Agrobacterium strain comprising each of the first, second and third plasmids of the system of claim 1.

13. The Agrobacterium of claim 12, wherein the Agrobacterium strain is selected from the group consisting of a nopaline synthesizing strain, a mannopine synthesizing strain, a succinamopine synthesizing strain, or an octopine synthesizing strain.

14. The Agrobacterium of claim 12, wherein the third plasmid is selected from the group consisting of pTiBo542, pTiC58, pTiAch5, a pTiChry5, or a derivative thereof.

15. The Agrobacterium of claim 12,
wherein the first VirD1 coding sequence comprises a polynucleotide that encodes a protein sequence with at least 97% sequence identity to SEQ ID NO:3;
wherein the first VirD2 coding sequence comprises a polynucleotide that encodes a protein sequence with at least 95% sequence identity to SEQ ID NO:14;
wherein the second VirD1 coding sequence comprises a polynucleotide that encodes a protein sequence with at least 97% sequence identity to SEQ ID NO:8; and
wherein the second VirD2 coding sequence comprises a polynucleotide that encodes a protein sequence with at least 95% sequence identity to SEQ ID NO:22.

16. The Agrobacterium of claim 12, wherein the T-DNA border sequence is selected from the group consisting of SEQ ID NO:28, SEQ ID NO:29, SEQ ID NO:30, SEQ ID NO:31, SEQ ID NO:32, and SEQ ID NO:33.

17. The Agrobacterium of claim 12, wherein the T-DNA border sequence comprises a polynucleotide sequence with at least 95% sequence identity to SEQ ID NO:29 or at least 95% sequence identity to SEQ ID NO:31.

18. The Agrobacterium strain of claim 12, wherein the T-DNA region comprises a gene expression cassette.

19. A transgenic plant or plant cell produced by
a. contacting plant cells with the Agrobacterium strain of claim 12;
b. selecting for plant cells that have said T-DNA integrated into plant genome; and
c. regenerating one or more transgenic plants from said cells.

20. The transgenic plant or plant cell of claim 19, wherein the transgenic plant comprises a transgenic event and the transgenic event comprises an agronomic trait or an herbicide tolerant trait.
